(19) United States
(12) Patent Application Publication
(10) Pub. No.: US 2026/0261808 A1
(43) Pub. Date: Sep. 3, 2026

(54) HEARING ASSISTANCE METHOD AND APPARATUS, ELECTRONIC DEVICE, AND STORAGE MEDIUM

(71) Applicant: TENCENT TECHNOLOGY (SHENZHEN) COMPANY LIMITED, Shenzhen (CN)

(72) Inventors: Jun ZHAO, Shenzhen (CN); Chang LV, Shenzhen (CN); Guangqi ZHEN, Shenzhen (CN); Cong FU, Shenzhen (CN); Shidong SHANG, Shenzhen (CN)

(21) Appl. No.: 19/659,614

(22) Filed: Apr. 27, 2026

Related U.S. Application Data
(63) Continuation of application No. PCT/CN2025/080415, filed on Mar. 4, 2025.

(30) Foreign Application Priority Data
Mar. 28, 2024 (CN) 202410374380.9

(51) Int. Cl. H04R 25/00; A61B 5/12; H04L 5/14; H04S 1/00

(52) U.S. Cl. CPC H04R 25/505; A61B 5/123; H04L 5/14; H04R 25/554; H04R 25/558; H04R 25/70; H04S 1/007; H04R 2225/55

(57) ABSTRACT

A hearing assistance method includes: establishing an audio/video call between the hearing assistance terminal and an assistance receiving terminal by using call software; obtaining an execution instruction for a hearing assistance application integrated into the call software, and running the hearing assistance application in the call software according to the execution instruction; establishing a data transmission channel between the hearing assistance terminal and the assistance receiving terminal based on the hearing assistance application, the data transmission channel being a duplex communication channel for transmitting application data of the hearing assistance application; and providing a hearing assistance service to the assistance receiving terminal through the data transmission channel and the audio/video call.

### CROSS-REFERENCES TO RELATED APPLICATIONS

This application is a continuation of PCT Application No. PCT/CN2025/080415, filed on Mar. 4, 2025, which claims priority to Chinese Patent Application No. 202410374380.9, filed on Mar. 28, 2024 and entitled “HEARING ASSISTANCE METHOD AND APPARATUS, ELECTRONIC DEVICE, AND STORAGE MEDIUM”, the entire contents of all of which are incorporated herein by reference.

### FIELD OF THE TECHNOLOGY

The present disclosure relates to the field of computers, and specifically, to a hearing assistance method and apparatus, an electronic device, and a storage medium.

### BACKGROUND OF THE DISCLOSURE

For hearing impaired people, proper use of hearing aids, cochlear implants, and other assistive listening devices (also called hearing assistance devices) is an effective means of rehabilitation, and can greatly help improve quality of life. A hearing assistance parameter of a hearing assistance device needs to be configured according to a hearing status of a hearing impaired person. Besides, after the hearing assistance device is worn, the hearing assistance parameter of the hearing assistance device further needs to be adjusted according to an actual wearing status. A hearing test on a hearing impaired person, configuration of a hearing assistance device usually need participation of professionals. Due to transportation costs, inconvenient travel of the hearing impaired person, or the like, costs of receiving, by the hearing impaired person, a hearing assistance service by a professional are relatively high. In addition, an application for implementing a remote hearing assistance service in the related technology has problems such as instability, difficulty in development, and inconvenient communication between the hearing impaired person and the professional, reducing quality of the hearing assistance service.

### SUMMARY

Embodiments of the present disclosure provide a hearing assistance method and apparatus, an electronic device, and a storage medium.

According to an aspect of the embodiments of the present disclosure, a hearing assistance method is provided, executed by a hearing assistance terminal. The method includes: establishing an audio/video call between the hearing assistance terminal and an assistance receiving terminal by using call software; obtaining an execution instruction for a hearing assistance application integrated into the call software, and running the hearing assistance application in the call software according to the execution instruction; establishing a data transmission channel between the hearing assistance terminal and the assistance receiving terminal based on the hearing assistance application, the data transmission channel being a duplex communication channel for transmitting application data of the hearing assistance application; and providing a hearing assistance service to the assistance receiving terminal through the data transmission channel and the audio/video call.

According to an aspect of the embodiments of the present disclosure, a hearing assistance method is provided, executed by an assistance receiving terminal. The method includes: establishing an audio/video call between the assistance receiving terminal and a hearing assistance terminal by using call software; obtaining an execution instruction for a hearing assistance application integrated into the call software, and running the hearing assistance application in the call software according to the execution instruction; establishing a data transmission channel between the assistance receiving terminal and the hearing assistance terminal based on the hearing assistance application, the data transmission channel being a duplex communication channel for transmitting application data of the hearing assistance application; and obtaining, through the data transmission channel and the audio/video call, a hearing assistance service provided by the hearing assistance terminal.

According to an aspect of the embodiments of the present disclosure, a hearing assistance apparatus is provided. The apparatus includes: an establishment module, configured to establish an audio/video call between a hearing assistance terminal and an assistance receiving terminal by using call software; a running module, configured to obtain an execution instruction for a hearing assistance application integrated into the call software, and run the hearing assistance application in the call software according to the execution instruction; a communications module, configured to establish a data transmission channel between the hearing assistance terminal and the assistance receiving terminal based on the hearing assistance application, the data transmission channel being a duplex communication channel for transmitting application data of the hearing assistance application; and a service module, configured to provide a hearing assistance service to the assistance receiving terminal through the data transmission channel and the audio/video call.

According to an aspect of the embodiments of the present disclosure, an electronic device is provided, including: one or more processors; and a storage apparatus, configured to store one or more computer programs, the one or more computer programs, when executed by the one or more processors, causing the electronic device to implement the method described above.

According to an aspect of the embodiments of the present disclosure, a non-transitory computer-readable storage medium is provided, having a computer program stored therein, the computer program, when executed by a processor of an electronic device, causing the electronic device to implement the method described above.

Details of one or more embodiments of the present disclosure are provided in the accompanying drawings and descriptions below. Other features, objectives, and advantages of the present disclosure become apparent from the specification, the accompanying drawings, and the claims.

### BRIEF DESCRIPTION OF THE DRAWINGS

To describe the technical solutions in the embodiments of the present disclosure or in the conventional technology more clearly, the following briefly describes the accompanying drawings required for describing the embodiments. Apparently, the accompanying drawings in the following description show only the embodiments of the present disclosure.

FIG. 1 is a schematic diagram of an implementation environment according to an exemplary embodiment of the present disclosure;

FIG. 2 is a flowchart of a hearing assistance method according to an exemplary embodiment of the present disclosure;

FIG. 3 is a diagram of an interface of an application store according to an exemplary embodiment of the present disclosure;

FIG. 4 is a flowchart of a hearing assistance method according to another exemplary embodiment of the present disclosure;

FIG. 5 is a diagram of a parameter setting interface according to an exemplary embodiment of the present disclosure;

FIG. 6 is a schematic diagram of a feedback interface according to another exemplary embodiment of the present disclosure;

FIG. 7 is a schematic diagram of a hearing test result according to another exemplary embodiment of the present disclosure;

FIG. 8 is a flowchart of a hearing assistance method according to another exemplary embodiment of the present disclosure;

FIG. 9 is a flowchart of a hearing assistance method according to another exemplary embodiment of the present disclosure;

FIG. 10 is a flowchart of a hearing assistance method according to another exemplary embodiment of the present disclosure;

FIG. 11 is a flowchart of a hearing assistance method according to another exemplary embodiment of the present disclosure;

FIG. 12 is a flowchart of a hearing assistance method according to another exemplary embodiment of the present disclosure;

FIG. 13 is a schematic diagram of a process of calculating a hearing assistance parameter according to another exemplary embodiment of the present disclosure;

FIG. 14 is a flowchart of a hearing assistance method according to another exemplary embodiment of the present disclosure;

FIG. 15 is a schematic diagram of an interface for adjusting a hearing assistance parameter according to another exemplary embodiment of the present disclosure;

FIG. 16 is a flowchart of a hearing assistance method according to another exemplary embodiment of the present disclosure;

FIG. 17 is a flowchart of a hearing assistance method according to another exemplary embodiment of the present disclosure;

FIG. 18 is a flowchart of a hearing assistance method according to another exemplary embodiment of the present disclosure;

FIG. 19 is a flowchart of a hearing assistance method according to another exemplary embodiment of the present disclosure;

FIG. 20 is a flowchart of a hearing assistance method according to another exemplary embodiment of the present disclosure;

FIG. 21 is a schematic diagram of an implementation environment according to another exemplary embodiment of the present disclosure;

FIG. 22 is a schematic flowchart of hearing assistance according to another exemplary embodiment of the present disclosure;

FIG. 23 is a schematic diagram of an invitation interface according to another exemplary embodiment of the present disclosure;

FIG. 24 is a flowchart of a channel creation process according to another exemplary embodiment of the present disclosure;

FIG. 25 is a schematic diagram of a service selection interface according to another exemplary embodiment of the present disclosure;

FIG. 26 is a flowchart of a hearing test process according to another exemplary embodiment of the present disclosure;

FIG. 27 is a flowchart of a hearing test process according to an exemplary embodiment of the present disclosure;

FIG. 28 is a schematic diagram of a selection interface according to an exemplary embodiment of the present disclosure;

FIG. 29 is a flowchart of a parameter configuration process according to an exemplary embodiment of the present disclosure;

FIG. 30 is a flowchart of a parameter adjustment process according to an exemplary embodiment of the present disclosure;

FIG. 31 is a structural diagram of a hearing assistance apparatus according to an exemplary embodiment of the present disclosure;

FIG. 32 is a structural diagram of a hearing assistance apparatus according to an exemplary embodiment of the present disclosure; and

FIG. 33 is a schematic structural diagram of a computer system configured to implement an electronic device according to an embodiment of the present disclosure.

### DESCRIPTION OF EMBODIMENTS

The technical solutions in embodiments of the present disclosure are clearly and completely described in the following with reference to the accompanying drawings in the embodiments of the present disclosure. Apparently, the described embodiments are merely some rather than all of the embodiments of the present disclosure. All other embodiments obtained by a person of ordinary skill in the art based on the embodiments of the present disclosure without creative efforts shall fall within the protection scope of the present disclosure.

Exemplary implementations are now described in a more comprehensive manner with reference to the accompanying drawings. However, the exemplary implementations can be implemented in various forms, and are not to be understood as being limited to the examples. On the contrary, the implementations are provided to make the present disclosure more thorough and complete, and fully convey the idea of the exemplary implementations to a person skilled in the art.

In addition, the features, structures or characteristics described in the present disclosure may be combined in one or more embodiments in any appropriate manner. The following description has many specific details, so that the embodiments of the present disclosure can be fully understood. However, a person skilled in the art is to be aware that, technical solutions of the present disclosure may be implemented without using all detailed features in the embodiments, one or more particular details may be omitted, or other methods, elements, apparatuses, or operations may be used.

In the embodiment of the present disclosure, the term “module” or “unit” refers to a computer program with a preset function or a part of the computer program and works, together with other related parts, to implement a preset target, and may be completely or partially implemented by using software, hardware (for example, a processing circuit or a memory) or a combination thereof. Similarly, one processor (or a plurality of processors or memories) may be configured to implement one or more modules or units. In addition, each module or unit may be a part of an overall module or unit including a function of the module or unit.

The block diagrams shown in the accompanying drawings are merely functional entities and do not necessarily correspond to physically independent entities. That is, the functional entities may be implemented in a software form, or in one or more hardware modules or integrated circuits, or in different networks and/or processor apparatuses and/or microcontroller apparatuses.

The flowcharts shown in the accompanying drawings are merely exemplary descriptions, do not need to include all content and operations, and do not need to be performed in the described orders either. For example, some operations may be further divided, while some operations may be combined or partially combined. Therefore, an actual execution order may change according to an actual case.

The term “plurality of” mentioned in the specification means two or more. “And/or” describes an association relationship for describing associated objects and represents that three relationships may exist. For example, A and/or B may represent the following three cases: Only A exists, both A and B exist, and only B exists. The character “/” generally indicates an “or” relationship between the associated objects.

The technical solutions of the embodiments of the present disclosure are described in detail in the following.

In the related technology, costs of receiving, by a hearing impaired person, a hearing assistance service by a professional are relatively high. In addition, an application for implementing a remote hearing assistance service has problems such as instability, difficulty in development, and inconvenient communication between the hearing impaired person and the professional. Based on this, the embodiments of the present disclosure provide a hearing assistance method and apparatus, an electronic device, a storage medium, and a program product, to improve hearing assistance service quality.

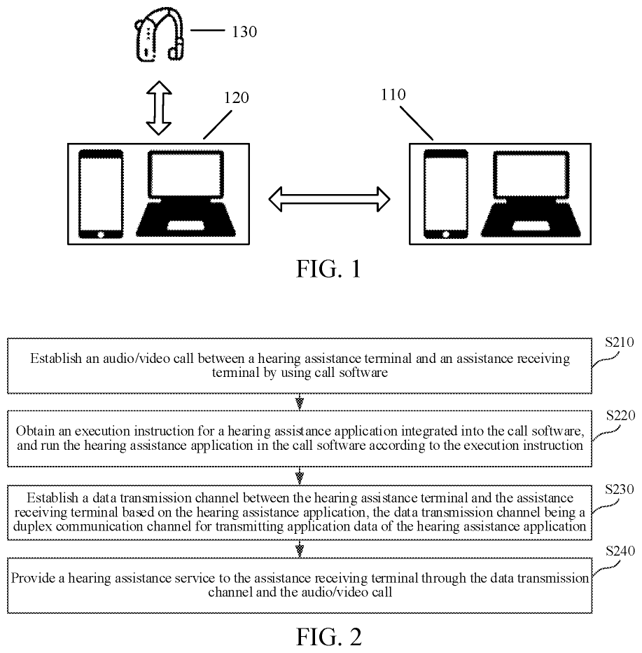

FIG. 1 is a schematic diagram of an implementation environment according to the present disclosure. The implementation environment includes a hearing assistance terminal 110 and an assistance receiving terminal 120. The hearing assistance terminal 110 communicates with the assistance receiving terminal 120 by using a wired or wireless network. The hearing assistance terminal 110 may send data to the assistance receiving terminal 120, or may obtain data from the assistance receiving terminal 120.

The hearing assistance terminal 110 includes, but is not limited to, at least one of a terminal device and a server, refers to a terminal that provides a hearing assistance service, and may be a device corresponding to a hearing assistance provider. The hearing assistance provider is configured to provide a hearing assistance service to a hearing impaired patient, for example, may be an audiologist or a doctor. The assistance receiving terminal 120 includes, but is not limited to, at least one of a terminal device and a server, is a terminal that obtains the hearing assistance service, and may be a device corresponding to a hearing assistance receiving party. The hearing assistance receiving party is an object to which the hearing assistance service needs to be provided, for example, may be a hearing impaired patient. The terminal device may include, but is not limited to, a mobile phone, a tablet computer, a laptop computer, a computer, a voice interaction device, a home appliance, an in-vehicle terminal, an aircraft, a remote driving terminal, a payment device (for example, a palm scanning device), and the like. The server may be an independent physical server, or a server cluster or a distributed system including multiple physical servers, or a cloud server providing basic cloud computing services such as a cloud service, a cloud database, cloud computing, a cloud function, cloud storage, a network service, cloud communication, a middleware service, a domain name service, a security service, a content delivery network (CDN), and a big data and artificial intelligence platform. Specific forms of the terminal device and the server are not limited herein.

Quantities of hearing assistance terminals 110 and assistance receiving terminals 120 in FIG. 1 are merely exemplary, and there may be any quantity of hearing assistance terminals 110 and assistance receiving terminals 120 according to an actual requirement.

In an exemplary embodiment, the hearing assistance terminal 110 and the assistance receiving terminal 120 provided in this embodiment of the present disclosure are jointly executed. Exemplarily, the hearing assistance terminal 110 may establish an audio/video call between the hearing assistance terminal 110 and the assistance receiving terminal 120 by using call software, obtain an execution instruction for a hearing assistance application integrated into the call software, run the hearing assistance application in the call software according to the execution instruction, and then establish, based on the hearing assistance application, a data transmission channel between the hearing assistance terminal 110 and the assistance receiving terminal 120. The data transmission channel is a duplex communication channel for transmitting application data of the hearing assistance application. A hearing assistance service is provided to the assistance receiving terminal 120 through the data transmission channel and the audio/video call. Compared with the related technology, a hearing assistance provider corresponding to the hearing assistance terminal 110 can communicate with a hearing assistance receiving party corresponding to the assistance receiving terminal 120 through the audio/video call, thereby improving convenience and accuracy of the hearing assistance service. In addition, the audio/video call is established based on call software, and stability of the audio/video call may be improved. The hearing assistance application does not need to integrate an audio/video call function. In addition, the hearing assistance application is integrated into the call software and runs based on the call software, which can reduce difficulty in developing the hearing assistance application, improve stability of the hearing assistance application, and improve quality of the hearing assistance service.

In an embodiment, the implementation environment may further include a hearing assistance device 130, configured to improve hearing of a hearing assistance receiving party. The hearing assistance receiving party can hear more sound based on the hearing assistance device 130 after wearing the hearing assistance device 130. A type of the hearing assistance device 130 includes, but is not limited to, a hearing aid and a cochlear implant. The hearing assistance device 130 may be connected to the assistance receiving terminal 120 based on a near field communication protocol such as Bluetooth. The hearing assistance terminal 110 may control the hearing assistance device 130, for example, configure a hearing assistance parameter of the hearing assistance device 130, through the data transmission channel, the audio/video call, and the connection between the assistance receiving terminal 120 and the hearing assistance device 130.

Data related to a user such as a hearing test result is subsequently involved in the embodiments of the present disclosure. When the method of the present disclosure is applied to a specific product or technology, user permission or agreement is obtained, and extraction, use, and processing of related data comply with local security standards and local regulations.

FIG. 2 is a flowchart of a hearing assistance method according to an exemplary embodiment of the present disclosure. The method may be applied to the implementation environment shown in FIG. 1, and may be performed by the hearing assistance terminal 110 in the implementation environment shown in FIG. 1.

As shown in FIG. 2, in an exemplary embodiment, the hearing assistance method may include operation S210 to operation S240, which are described in detail as follows:
Operation S210: Establish an audio/video call between the hearing assistance terminal and an assistance receiving terminal by using call software.

The hearing assistance terminal is a terminal that provides a hearing assistance service, includes, but is not limited to, at least one of a terminal device and a server, and may be a device corresponding to a hearing assistance provider. The hearing assistance provider is configured to provide a hearing assistance service for a hearing impaired patient, for example, a professional that can provide a hearing assistance service, such as an audiologist or a doctor. A user may run a hearing assistance application in the device, and select a hearing assistance provider mode in the hearing assistance application, so that the device is used as a hearing assistance terminal for providing a hearing assistance service.

A hearing assistance receiving terminal is a terminal that obtains a hearing assistance service, includes, but is not limited to, at least one of a terminal device and a server, and may be a device corresponding to a hearing assistance receiving party. The hearing assistance receiving party is an object to which the hearing assistance service needs to be provided, for example, a hearing impaired patient. The hearing assistance receiving party is an object to which the hearing assistance service needs to be provided, for example, a hearing impaired patient. A user may run a hearing assistance application in the device, and select a hearing assistance receiving party mode in the hearing assistance application, so that the device is used as the assistance receiving terminal. The hearing assistance receiving party may wear a hearing assistance device. The hearing assistance device is configured to improve hearing of a hearing assistance receiving party. Types of the hearing assistance device include, but are not limited to, a hearing aid, a cochlear implant, and the like. The hearing assistance device may be connected to an assistance receiving terminal based on a near field communication protocol such as Bluetooth. A hearing assistance parameter of the hearing assistance device needs to be configured and adjusted according to a hearing status of a hearing impaired person.

The call software is software having an audio/video call function. The audio/video call includes, but is not limited to, a voice call, a video call, and the like. Correspondingly, the call software includes, but is not limited to, an application that can provide a voice call and video call function, for example, an application that can provide an audio/video call function, such as an online conferencing application and a social chat application.

Call software is installed in both the hearing assistance terminal and the assistance receiving terminal. By using the call software, an audio/video call between the hearing assistance terminal and the assistance receiving terminal may be established, so that a hearing assistance provider and a hearing assistance receiving party may perform face-to-face communication and the like based on the call. A specific manner for establishing the audio/video call may be set flexibly according to an actual requirement. In an exemplary embodiment, if the call software is a social chat application, the audio/video call may be initiated by the hearing assistance terminal. Correspondingly, the hearing assistance provider may open the social chat application installed in the hearing assistance terminal, and initiate an audio/video call request to the assistance receiving terminal according to accounts of the hearing assistance provider and the hearing assistance receiving party in the social chat application. If the hearing assistance receiving party chooses to answer the audio/video call in the assistance receiving terminal, an audio/video call is established between the hearing assistance terminal and the assistance receiving terminal. Alternatively, the audio/video call may be initiated by the assistance receiving terminal. Correspondingly, the hearing assistance receiving party may open a social chat application installed in the assistance receiving terminal, and initiate an audio/video call request to the hearing assistance terminal according to accounts of the hearing assistance provider and the hearing assistance receiving party in the social chat application. If the hearing assistance provider chooses to answer the audio/video call in the hearing assistance terminal, the audio/video call between the hearing assistance terminal and the assistance receiving terminal is established. In another exemplary embodiment, if an audio/video call service is an online conferencing application, the hearing assistance provider and the hearing assistance receiving party may agree on a conference time, and apply for a conference number in the online conferencing application according to the conference time. Subsequently, the hearing assistance provider may join the conference by using the online conferencing application in the hearing assistance terminal, and the hearing assistance receiving party may join the conference by using the online conferencing application in the assistance receiving terminal, so as to establish the audio/video call.
Operation S220: Obtain an execution instruction for a hearing assistance application integrated into the call software, and run the hearing assistance application in the call software according to the execution instruction.

The hearing assistance application is an application configured for providing a hearing assistance service, for example, for providing a hearing assistance service such as a hearing test service, a matching service, and an information management service. The matching service is configured to update a hearing assistance parameter of a hearing assistance device, and includes, but is not limited to, a parameter configuration service and a parameter adjustment service. The parameter configuration service is configured for configuring the hearing assistance parameter of the hearing assistance device. The parameter adjustment service is configured for adjusting the hearing assistance parameter of the hearing assistance device. For specific description of the hearing assistance parameter, refer to subsequent description, and details are not described herein again. The information management service is configured to manage attribute information of the hearing assistance device, for example, obtaining or configuring attribute information of the hearing assistance device. The attribute information of the hearing assistance device is information related to an attribute of the hearing assistance device, and includes, but is not limited to, a manufacturer, a form, a model, a battery amount, an operating mode, a supported communication protocol version, a hardware address, and the like of the hearing assistance device. The hearing assistance application provides a hearing assistance receiving party mode and a hearing assistance provider mode. Different modes provide different control permissions, functions, and the like. A user may select a mode to run the hearing assistance application. The hearing assistance terminal may run the hearing assistance application in the hearing assistance provider mode, and the assistance receiving terminal may run the hearing assistance application in the hearing assistance receiving party mode.

The hearing assistance application does not independently run, and instead is hosted in the call software and runs depending on the call software. That is, the call software is a host of the hearing assistance application. Hosting means that the hearing assistance application runs depending on the call software, and implements a function by using an environment, a component, or an interface provided by the call software. The call software is used as a host of the hearing assistance application, and provides a running basis for the hearing assistance application. For example, the hearing assistance application may be a mini program, and is hosted in the call software by using an interface. For another example, the hearing assistance application may be a plug-in, and is hosted in the call software by using a plug-in technology. For another example, the hearing assistance application may be a web application, and is hosted in the call software by using a browser component.

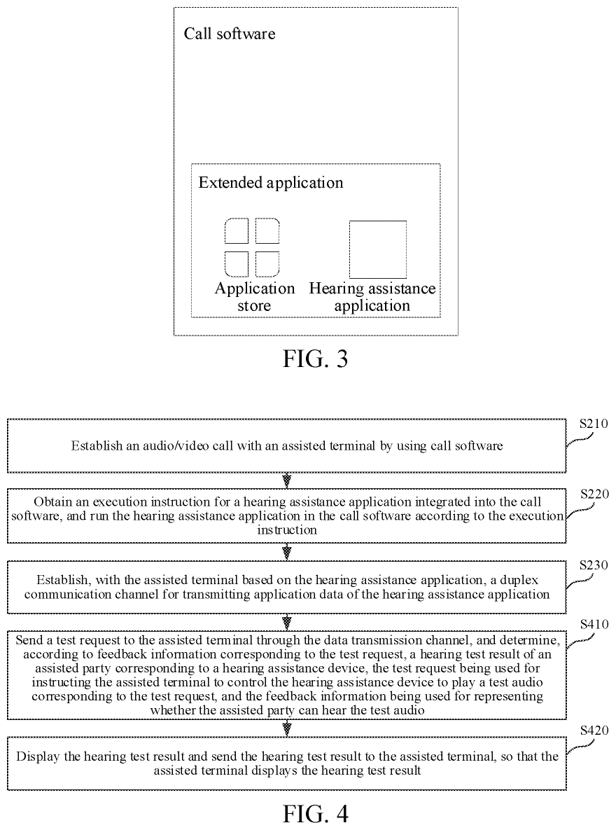

After the call software is opened, if a hearing assistance service needs to be provided to the assistance receiving terminal, an execution instruction for the hearing assistance application may be obtained, and the hearing assistance application may be run in the call software according to the execution instruction. The execution instruction may be triggered by the hearing assistance provider. That is, the hearing assistance provider may find the hearing assistance application in the call software, open the hearing assistance application, and select the hearing assistance provider mode. In an embodiment, referring to FIG. 3, an application store of the call software may be searched for the hearing assistance application.
Operation S230: Establish a data transmission channel between the hearing assistance terminal and the assistance receiving terminal based on the hearing assistance application, the data transmission channel being a duplex communication channel for transmitting application data of the hearing assistance application.

To implement the remote hearing assistance service by using the hearing assistance application, a data transmission channel between the hearing assistance terminal and the assistance receiving terminal may be established based on the hearing assistance application. The data transmission channel is a duplex communication channel for transmitting application data of the hearing assistance application. Subsequently, the application data corresponding to the hearing assistance application may be transmitted between the hearing assistance terminal and the assistance receiving terminal through the data transmission channel. A type of the established data transmission channel may be flexibly set according to an actual requirement, and in an exemplary embodiment, may be a point-to-point protocol channel. The application data of the hearing assistance application includes, but is not limited to, data generated by the hearing assistance application, data received by the hearing assistance application, and data transmitted by the hearing assistance application, for example, a subsequent test request and a parameter configuration policy.

The data transmission channel is a duplex communication channel that is established based on the hearing assistance application and that is configured for transmitting application data of the hearing assistance application. Data may be transmitted between the hearing assistance terminal and the assistance receiving terminal. A type of the data transmission channel may be set flexibly according to an actual requirement, for example, a point-to-point protocol channel. Application data is data generated by the hearing assistance application in a running process, received data, transmitted data, or the like, for example, a test request and a parameter configuration policy, and may be transmitted between the hearing assistance terminal and the assistance receiving terminal through the data transmission channel.

In an exemplary embodiment, the hearing assistance provider may trigger, in the hearing assistance terminal, an invitation request for inviting the assistance receiving terminal to perform the hearing assistance service. The hearing assistance terminal may send the invitation request to the assistance receiving terminal. After the assistance receiving terminal receives the invitation request, the hearing assistance receiving party may trigger an operation of agreeing to the invitation request. The assistance receiving terminal generates an invitation agreement response based on the operation, so as to create a data transmission channel between the hearing assistance terminal and the assistance receiving terminal based on the invitation agreement response. The data transmission channel is a duplex communication channel for transmitting application data of the hearing assistance application. In the foregoing example, the invitation request is triggered by the hearing assistance provider in the hearing assistance terminal. In another exemplary embodiment, the invitation request may be triggered by the hearing assistance receiving party in the assistance receiving terminal, to invite the hearing assistance terminal to perform the hearing assistance service.
Operation S240: Provide a hearing assistance service to the assistance receiving terminal through the data transmission channel and the audio/video call.

After the data transmission channel and the audio/video call are established, the hearing assistance receiving party and the hearing assistance provider may communicate with each other by using the audio/video call. Meanwhile, the application data of the hearing assistance application is transmitted between the hearing assistance terminal and the assistance receiving terminal through the data transmission channel, to transmit data triggered by the hearing assistance provider or the hearing assistance receiving party in the hearing assistance application to the other party. Therefore, this implements a remote service of the hearing assistance application, and better provides the hearing assistance service to the hearing assistance receiving party.

In the embodiment shown in FIG. 2, the application data of the hearing assistance application is transmitted between the assistance receiving terminal and the hearing assistance terminal by using the data transmission channel, so that the remote service of the hearing assistance application is implemented. The audio/video call implements communication between the hearing assistance provider corresponding to the hearing assistance terminal and the hearing assistance receiving party corresponding to the assistance receiving terminal, thereby improving convenience and accuracy of the hearing assistance service. In addition, the audio/video call is established based on the call software, so that stability of the audio/video call can be improved. The hearing assistance application does not need to integrate an audio/video call function, and the hearing assistance application is hosted in the call software and runs based on the call software. This can reduce difficulty in developing the hearing assistance application, improve stability of the hearing assistance application, and improve quality of the hearing assistance service.

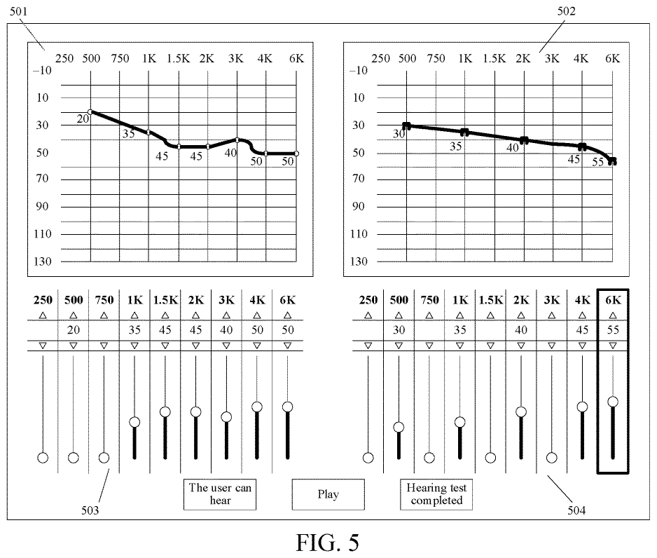

In an exemplary embodiment, FIG. 4 is a flowchart of a hearing assistance method based on FIG. 2. The method may be applied to the implementation environment shown in FIG. 1, and may be performed by the hearing assistance terminal 110 in the implementation environment shown in FIG. 1.

As shown in FIG. 4, operation S240 may include operation S410 and operation S420, and details are described as follows:
Operation S410: Send a test request to the assistance receiving terminal through the data transmission channel, and determine, according to feedback information corresponding to the test request, a hearing test result of a hearing assistance receiving party corresponding to a hearing assistance device, the test request being configured to instruct the assistance receiving terminal to control the hearing assistance device to play a test audio corresponding to the test request, and the feedback information representing whether the hearing assistance receiving party can hear the test audio.

The hearing assistance service is a series of services provided to the hearing assistance receiving party, including a hearing test service, a matching service configured to update a hearing assistance parameter of a hearing assistance device (for example, a parameter configuration service and a parameter adjustment service), an information management service configured to manage attribute information of the hearing assistance device, and the like.

The hearing assistance service may include a hearing test service configured for testing a hearing status of the hearing assistance receiving party. The test request is used to instruct the assistance receiving terminal to control the hearing assistance device to play a test audio corresponding to the test request. When a hearing test needs to be performed, the hearing assistance terminal may generate the test request, and send the test request to the assistance receiving terminal. The assistance receiving terminal is connected to the hearing assistance device. After receiving the test request, the assistance receiving terminal may determine a corresponding test audio according to the test request, and control the hearing assistance device to play the test audio, so as to test whether a person (that is, the hearing assistance receiving party) wearing the hearing assistance device can hear the test audio. Then, the hearing assistance device may obtain feedback information corresponding to the test request, where the feedback information represents whether the hearing assistance receiving party can hear the test audio corresponding to the test request, so as to determine a hearing test result of the hearing assistance receiving party according to the feedback information.

In an embodiment, the test request includes related data of the corresponding test audio, so that the assistance receiving terminal can determine the test audio according to the related data. The test request may directly include the test audio, or the test request may include a test sound frequency and a test sound intensity, so that an audio matching the test sound frequency and the test sound intensity is used as the test audio corresponding to the test request. The test sound frequency is a sound frequency configured for performing a hearing test, and a hearing loss degree of the hearing assistance receiving party at the sound frequency needs to be tested. The test sound intensity is a sound intensity configured for performing a hearing test, and it needs to be tested whether the hearing assistance receiving party can hear a sound of the sound intensity. In an exemplary embodiment, the test request may be automatically generated by the hearing assistance application. That is, the hearing assistance application provides an automatic test service. If the hearing assistance provider triggers the automatic test service in the hearing assistance application run on the hearing assistance terminal, the automatic test service may automatically determine related data of the test audio, and automatically generate the test request according to the related data. In another exemplary embodiment, the test request may be triggered by the hearing assistance provider. That is, the hearing assistance application provides a manual test service. If the hearing assistance provider triggers the manual test service in the hearing assistance application run on the hearing assistance terminal, the hearing assistance terminal may obtain related data of the test audio set by the hearing assistance provider, and generate the test request according to the related data. For example, in an exemplary embodiment, FIG. 5 is an exemplary interface for setting a test parameter. The interface includes a left-ear hearing test result diagram 501 and a right-ear hearing test result diagram 502 of a hearing assistance receiving party. The test result diagram includes hearing thresholds of the hearing assistance receiving party at different sound frequencies. The interface further includes a left-ear hearing test parameter setting control 503 and a right-ear hearing test parameter setting control 504. After setting a test sound frequency of 6 KHz and a test sound intensity of 55 db in the control 504, the hearing assistance provider may trigger, by using a “play” control, the hearing assistance device to play, to the right ear, a test audio whose sound frequency is 6 KHz and sound intensity is 55 db.

A specific manner in which the hearing assistance terminal obtains the feedback information corresponding to the test request may be set flexibly according to an actual requirement. For example, the manner includes, but is not limited to, at least one of the following manners:

In a first method, after the hearing assistance device plays the test audio, the hearing assistance receiving party may input the feedback information by using a user input interface provided by the assistance receiving party, and the assistance receiving terminal may obtain the feedback information inputted on the assistance receiving terminal, and send the feedback information to the hearing assistance device through the data transmission channel, so that the hearing assistance device obtains the feedback information of the test request.

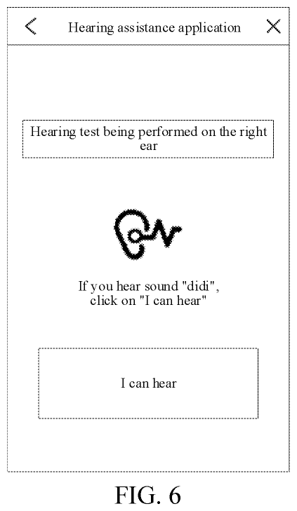

The user input interface may be a touch screen. Correspondingly, the hearing assistance application run on the assistance receiving terminal may display a feedback interface, and the hearing assistance receiving party may input the feedback information by using the feedback interface. For example, as shown in FIG. 6, the feedback interface includes a control “I heard”. After the test audio is played, if the hearing assistance receiving party can hear the test audio, the control “I can hear” may be triggered to input the feedback information representing that the hearing assistance receiving party can hear the test audio.

In an embodiment, after playing the test audio, if the assistance receiving terminal does not obtain, within a specified time period, the feedback information inputted by the hearing assistance receiving party, the assistance receiving terminal may alternatively generate feedback information representing that the hearing assistance receiving party cannot hear the test audio, and send the feedback information to the hearing assistance device through the data transmission channel.

In a second manner, the hearing assistance device displays a trigger control corresponding to the feedback information, obtains a trigger operation for the trigger control, and obtains the feedback information of the test request according to the trigger operation.

In an embodiment, the trigger control corresponding to the feedback information may be displayed in the hearing assistance application run in the hearing assistance device. After the test audio is played, the hearing assistance receiving party may feed back, to the hearing assistance provider by using the audio/video call, whether the test audio is heard. For example, the hearing assistance receiving party may express, to the hearing assistance provider by using a gesture, a speech, or the like, whether the test audio is heard. After obtaining the feedback of the user, the hearing assistance provider may trigger the displayed trigger control, so as to input the feedback information of the hearing assistance receiving party. For example, as shown in FIG. 5, the interface includes a control “the user can hear”. After the test audio is played, the hearing assistance provider may input, by triggering the control “the user can hear”, the feedback information representing that the hearing assistance receiving party can hear.
Operation S420: Display the hearing test result and send the hearing test result to the assistance receiving terminal, so that the assistance receiving terminal displays the hearing test result.

After obtaining the hearing test result of the hearing assistance receiving party, the hearing assistance terminal may display the hearing test result for the hearing assistance provider to view. The hearing test result may further be sent to the assistance receiving terminal, so that the assistance receiving terminal displays the hearing test result for the hearing assistance receiving party to view.

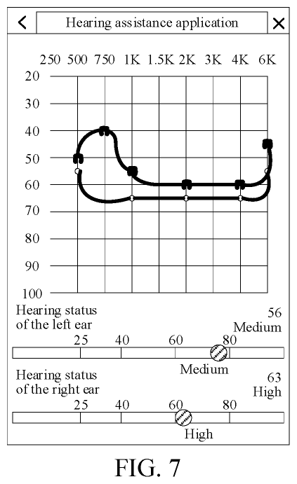

The hearing test result may include at least one of a left-ear hearing test result and a right-ear hearing test result of the hearing assistance receiving party. The hearing test result of each ear includes hearing thresholds of the ear at different sound frequencies. A hearing threshold refers to a minimum sound intensity that can be heard, and a hearing threshold at a sound frequency refers to a minimum sound intensity that can be heard at the sound frequency. The hearing test result may be displayed in a form of a graph. For example, FIG. 7 is an exemplary diagram displaying a hearing test result. A horizontal coordinate represents a sound frequency, and a vertical coordinate represents a sound intensity.

For specific implementation details of operation S210 to operation S230 shown in FIG. 4, refer to operation S210 to operation S230 shown in FIG. 2. Details are not described herein again.

In the embodiment shown in FIG. 4, the hearing test may be performed on the hearing assistance receiving party through the data transmission channel and the audio/video call, thereby improving accuracy and convenience of the hearing test.

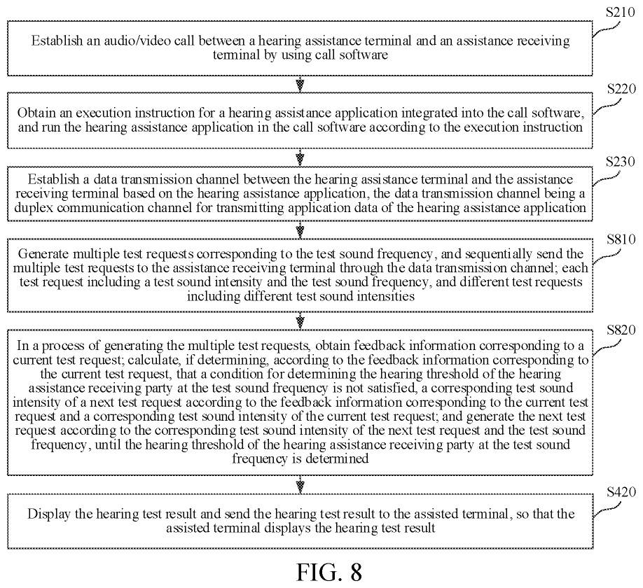

In an exemplary embodiment, FIG. 8 is a flowchart of a hearing assistance method based on FIG. 4. The method may be applied to the implementation environment shown in FIG. 1, and may be performed by the hearing assistance terminal 110 in the implementation environment shown in FIG. 1.

As shown in FIG. 8, if a hearing test result includes a hearing threshold of a hearing assistance receiving party at a specified test sound frequency, operation S410 may include operation S810 and operation S820, and details are described as follows:
Operation S810: Generate multiple test requests corresponding to the test sound frequency, and sequentially send the multiple test requests to the assistance receiving terminal through the data transmission channel; each test request including a test sound intensity and the test sound frequency, and different test requests including different test sound intensities.

When the hearing test result includes a hearing threshold of the hearing assistance receiving party at a specified test sound frequency, test audios of different sound intensities at the test sound frequency need to be played, so that the hearing threshold of the hearing assistance receiving party at the test sound frequency is determined according to feedback information of the hearing assistance receiving party for the test audios of different sound intensities. Therefore, multiple test requests corresponding to the test sound frequency may be generated, each test request includes the test sound frequency and a test sound intensity, and different test requests include different test sound intensities. The multiple test requests are sent to the assistance receiving terminal, so that the assistance receiving terminal controls, according to the multiple test requests, the hearing assistance device to play a test audios of different test sound intensities at the test sound frequency.
Operation S820: In a process of generating the multiple test requests, obtain feedback information corresponding to a current test request; calculate, in response to determining, according to the feedback information corresponding to the current test request, that a condition for determining the hearing threshold of the hearing assistance receiving party at the test sound frequency is not satisfied, a corresponding test sound intensity of a next test request according to the feedback information corresponding to the current test request and a corresponding test sound intensity of the current test request; and generate the next test request according to the corresponding test sound intensity of the next test request and the test sound frequency, until the hearing threshold of the hearing assistance receiving party at the test sound frequency is determined.

That the condition for determining the hearing threshold of the hearing assistance receiving party at the test sound frequency is not satisfied may be specifically: feedback information is insufficient, a test range is not covered, a sound intensity difference does not reach a threshold, playing of a test audio is problematic, or a state of the hearing assistance receiving party is unstable.

The feedback information being insufficient refers to that the feedback information cannot clearly indicate whether the hearing assistance receiving party can hear the test audio. For example, when a user inputs feedback information through an input interface, the feedback information may be ambiguous due to reasons such as mistaken operations or unclear expressions, and whether the user can hear a sound cannot be determined precisely. For example, the hearing assistance receiving party incorrectly touches another option when inputting the feedback information, and as a result, the feedback information received by the system is inconsistent with an actual case.

That the test range is not covered means that a sound intensity range that has been tested currently does not include the hearing threshold of the hearing assistance receiving party at the test sound frequency. For example, an initially set maximum sound intensity is excessively small or an initially set minimum sound intensity is excessively large, so that the hearing threshold is not within a currently tested sound intensity interval. If the initially set minimum sound intensity is 30 dB, but an actual hearing threshold may be 20 dB, the hearing threshold cannot be determined within the current test range.

That a sound intensity difference does not reach a threshold means that according to a manner of searching for a hearing threshold by using a bisection method in patents, when a difference between the updated maximum sound intensity and the updated minimum sound intensity is still greater than or equal to the specified intensity threshold, the hearing threshold cannot be determined. In this case, the search range further needs to be narrowed, and a further test is performed to approach the hearing threshold. For example, if the intensity threshold is set to 5 dB, but the current difference between the maximum sound intensity and the minimum sound intensity is 8 dB, the test sound intensity needs to be further adjusted to further test.

That playing of a test audio is problematic means that the test audio may encounter a problem during playing, such as a failure of the hearing assistance device or an audio transmission error. Consequently, the hearing assistance receiving party fails to normally receive the test audio, and therefore the hearing threshold cannot be determined according to feedback information thereof. For example, if a speaker of the hearing assistance device is damaged, and the test audio cannot be normally played, the hearing assistance receiving party naturally cannot hear the sound, and feedback information cannot reflect a real hearing status thereof.

That a state of the hearing assistance receiving party is unstable means that the state of the hearing assistance receiving party is unstable in a test process, for example, the hearing assistance receiving party is not focused or is physically uncomfortable, causing inaccurate feedback information. For example, the hearing assistance receiving party is suddenly interfered by the outside during the test and is not focused on hearing the test audio, and wrong feedback information may be provided, affecting determining of the hearing threshold.

In a process of generating the multiple test requests corresponding to the test sound frequency, a current test request may be sent to the assistance receiving terminal through the data transmission channel, so that the assistance receiving terminal plays a test audio matching the test sound frequency and the test sound intensity included in the current test request. The feedback information corresponding to the current test request is obtained, if it is determined, according to the feedback information corresponding to the current test request, that the condition for determining the hearing threshold of the hearing assistance receiving party at the test sound frequency is not satisfied, a corresponding test sound intensity of a next test request is calculated according to the feedback information and the corresponding test sound intensity of the current test request, the next test request is generated according to the test sound intensity and the test sound frequency corresponding to the next test request, the next test request is sent to the assistance receiving terminal through the data transmission channel, so that the assistance receiving terminal plays a test audio matching the test sound frequency and the test sound intensity included in the next test request, feedback information corresponding to the next test request is obtained, and so on, until the hearing threshold of the hearing assistance receiving party at the test sound frequency is determined. That is, from the perspective of a next test request, in a process of generating a test request other than the first test request of the multiple test requests corresponding to the test sound frequency, a test sound intensity included in this test request is automatically calculated according to a test sound intensity included in a previous test request of this test request and feedback information of the previous test request.

A specific manner of generating the multiple test requests corresponding to the test sound frequency may be flexibly set according to an actual requirement. In an exemplary embodiment, multiple different test sound intensities may be preset, and a first test sound intensity is extracted from the multiple test sound intensities according to an ascending order of the corresponding sound intensities, to generate a first test request. If feedback information of the first test request is that the user cannot hear, a second test sound intensity is extracted from the multiple test sound intensities, to generate a second test request. If feedback information of the second test request is that the user cannot hear, a third test sound intensity is extracted from the multiple test sound intensities, to generate a third test request, and so on, until a test request whose corresponding feedback information is that the user can hear is found, and a test sound intensity corresponding to this test request is used as the hearing threshold of the hearing assistance receiving party at the test sound frequency. Alternatively, a first test sound intensity may be extracted from the multiple test sound intensities according to a descending order of the corresponding sound intensities, to generate a first test request. If feedback information of the first test request is that the user can hear, a second test sound intensity is extracted from the multiple test sound intensities, to generate a second test request. If feedback information of the second test request is that the user can hear, a third test sound intensity is extracted from the multiple test sound intensities, to generate a third test request, and so on, until a test request whose corresponding feedback information is that the user cannot hear is found. A test sound intensity corresponding to a previous test request of this test request is used as the hearing threshold of the hearing assistance receiving party at the test sound frequency.
Operation S810 and operation S820 may be used to detect hearing thresholds of a hearing assistance receiving party at multiple test sound frequencies. According to a descending (or ascending) order of corresponding sound frequencies, after a hearing threshold of the hearing assistance receiving party at a current test sound frequency is detected, a hearing threshold of the hearing assistance receiving party at a next test sound frequency is detected. For specific implementation details of operation S210 to operation S230 shown in FIG. 8, refer to operation S210 to operation S230 shown in FIG. 2. For specific implementation details of operation S420 shown in FIG. 8, refer to operation S420 shown in FIG. 4. Details are not described herein again.

In the embodiment shown in FIG. 8, the hearing of the hearing assistance receiving party is automatically tested. Besides, a corresponding test sound intensity of a next test request may be calculated according to the feedback information and the corresponding test sound intensity of the current test request, thereby improving the hearing test efficiency.

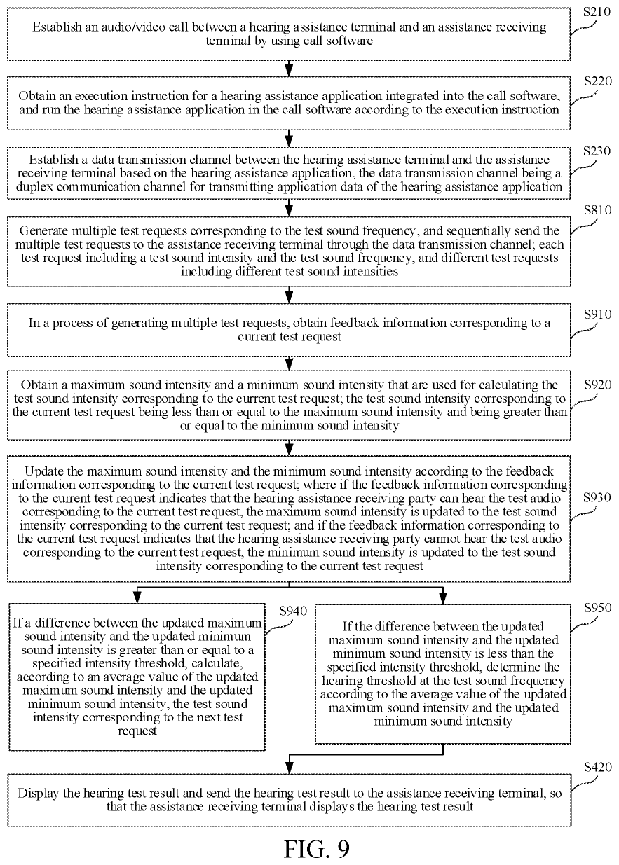

In an exemplary embodiment, FIG. 9 is a flowchart of a hearing assistance method based on FIG. 8. The method may be applied to the implementation environment shown in FIG. 1, and may be performed by the hearing assistance terminal 110 in the implementation environment shown in FIG. 1.

As shown in FIG. 9, operation S820 may include operation S910 to operation S950, and details are described as follows:
Operation S910: In a process of generating multiple test requests, obtain feedback information corresponding to a current test request.

For a specific manner of obtaining the feedback information corresponding to the test request, refer to the foregoing example, and details are not described herein again.
Operation S920: Obtain a maximum sound intensity and a minimum sound intensity that are configured for calculating the corresponding test sound intensity of the current test request; the corresponding test sound intensity of the current test request being less than or equal to the maximum sound intensity and being greater than or equal to the minimum sound intensity.

The test sound intensity of the current test request is calculated according to the maximum sound intensity and the minimum sound intensity, where the test sound intensity is less than or equal to the maximum sound intensity and is greater than or equal to the minimum sound intensity, and the hearing threshold of the hearing assistance receiving party is also less than or equal to the maximum sound intensity and is greater than or equal to the minimum sound intensity.

A maximum sound intensity corresponding to the first test request of the multiple test requests corresponding to the test sound frequency is a specified maximum sound intensity corresponding to the test sound frequency, and a corresponding minimum sound intensity is a minimum sound intensity corresponding to the test sound frequency. The test sound intensity corresponding to the first test request may be a specified sound intensity, or may be calculated according to an average value of the maximum sound intensity and the minimum sound intensity.

A maximum sound intensity and a minimum sound intensity of a test request of the multiple test requests corresponding to the test sound frequency other than the first test request are values updated according to feedback information corresponding to a previous test request. If the feedback information corresponding to the previous test request indicates that the hearing assistance receiving party can hear the test audio corresponding to the test request, the maximum sound intensity is updated to the test sound intensity corresponding to the previous test request; and if the feedback information corresponding to the previous test request indicates that the hearing assistance receiving party cannot hear the test audio corresponding to the test request, the minimum sound intensity is updated to the test sound intensity corresponding to the previous test request.
Operation S930: Update the maximum sound intensity and the minimum sound intensity according to the feedback information corresponding to the current test request; where if the feedback information corresponding to the current test request indicates that the hearing assistance receiving party can hear the test audio corresponding to the current test request, the maximum sound intensity is updated to the corresponding test sound intensity of the current test request; and if the feedback information corresponding to the current test request indicates that the hearing assistance receiving party cannot hear the test audio corresponding to the current test request, the minimum sound intensity is updated to the corresponding test sound intensity of the current test request.

If the feedback information corresponding to the current test request indicates that the hearing assistance receiving party can hear the test audio corresponding to the current test request, it indicates that a hearing threshold of the hearing assistance receiving party at the test sound frequency is less than or equal to the corresponding test sound intensity of the current test request and is greater than or equal to the minimum sound intensity. Therefore, the maximum sound intensity is updated to the corresponding test sound intensity of the current test request, and the minimum sound intensity is not updated. If the feedback information corresponding to the current test request indicates that the hearing assistance receiving party cannot hear the test audio corresponding to the current test request, it indicates that a hearing threshold of the hearing assistance receiving party at the test sound frequency is greater than the corresponding test sound intensity of the current test request and is less than or equal to the maximum sound intensity. Therefore, the minimum sound intensity is updated to the corresponding test sound intensity of the current test request, and the maximum sound intensity is not updated.
Operation S940: If a difference between the updated maximum sound intensity and the updated minimum sound intensity is greater than or equal to a specified intensity threshold, calculate the corresponding test sound intensity of the next test request according to an average value of the updated maximum sound intensity and the updated minimum sound intensity.

If the difference between the updated maximum sound intensity and the updated minimum sound intensity is greater than or equal to the specified intensity threshold, the hearing threshold of the hearing assistance receiving party at the test sound frequency needs to be searched based on the updated maximum sound intensity and the updated minimum sound intensity. Therefore, the average value of the updated maximum sound intensity and the updated minimum sound intensity is calculated, the test sound intensity of the next test request is obtained according to the average value, and a next test request is generated according to the test sound intensity of the next test request. That is, in this embodiment, the hearing threshold of the hearing assistance receiving party is searched by using a bisection method.

In an embodiment, the average value of the updated maximum sound intensity and the updated minimum sound intensity may be directly used as the test sound intensity of the next test request. Alternatively, because the test sound intensity corresponding to the test request is used as the hearing threshold, to make a value of the hearing threshold be an integer multiple of a specified value, it may be determined whether the average value of the updated maximum sound intensity and the updated minimum sound intensity is an integer multiple of the specified value. If yes, the average value is used as the test sound intensity of the next test request. If not, a value that has a minimum difference with the average value and that is an integer multiple of the specified value is obtained, and the value is used as the test sound intensity of the next test request. A value that has a minimum difference with the average value and that is less than the average value and that is an integer multiple of the specified value may be obtained as the test sound intensity of the next test request. Alternatively, a value that has a minimum difference with the average value and that is greater than the average value and that is an integer multiple of the specified value may be obtained as the test sound intensity of the next test request. The specified value may be an intensity threshold, or the specified value may be different from the intensity threshold.
Operation S950: If the difference between the updated maximum sound intensity and the updated minimum sound intensity is less than the specified intensity threshold, determine the hearing threshold at the test sound frequency according to the average value of the updated maximum sound intensity and the updated minimum sound intensity.

If the difference between the updated maximum sound intensity and the updated minimum sound intensity is less than the specified intensity threshold, the hearing threshold at the test sound frequency may be determined according to the average value of the updated maximum sound intensity and the updated minimum sound intensity. For example, the corresponding test sound intensity of the next test request can be used as the hearing threshold, and no further test request is generated. That the difference between the updated maximum sound intensity and the updated minimum sound intensity is less than the specified intensity threshold is the condition for determining the hearing threshold of the hearing assistance receiving party at the test sound frequency.

For specific implementation details of operation S210 to operation S230 shown in FIG. 9, refer to operation S210 to operation S230 shown in FIG. 2. For specific implementation details of operation S420 shown in FIG. 9, refer to operation S420 shown in FIG. 4. For specific implementation details of operation S810 shown in FIG. 9, refer to operation S810 shown in FIG. 8. Details are not described herein again.

In the embodiment shown in FIG. 9, the hearing threshold of the hearing assistance receiving party at the test sound frequency is gradually determined by using a bisection method, so that hearing test efficiency can be improved.

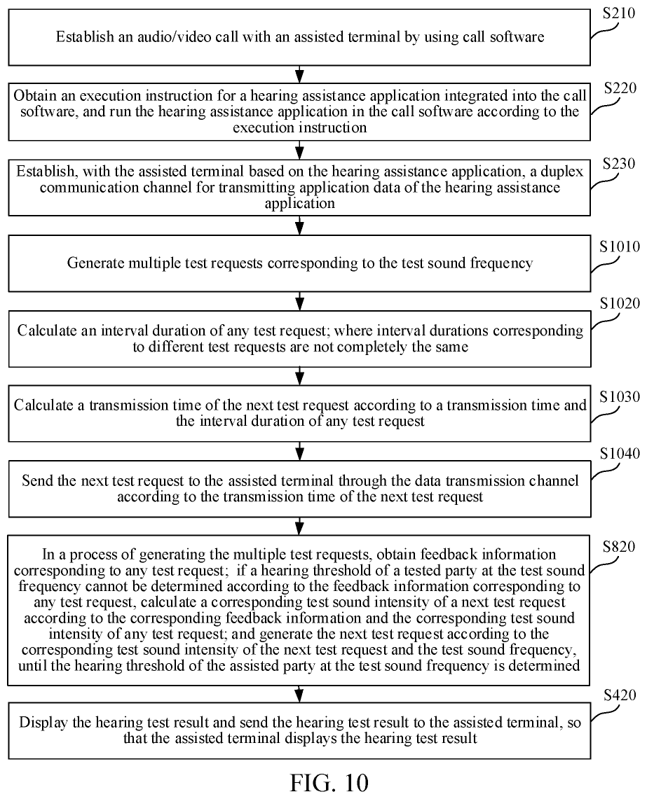

In an exemplary embodiment, FIG. 10 is a flowchart of a hearing assistance method based on FIG. 8. The method may be applied to the implementation environment shown in FIG. 1, and may be performed by the hearing assistance terminal 110 in the implementation environment shown in FIG. 1.

As shown in FIG. 10, operation S810 may include operation S1010 to operation S1040, and details are described as follows:
Operation S1010: Generate the multiple test requests corresponding to the test sound frequency.

For a specific process of generating the multiple test requests, refer to the foregoing descriptions, and details are not described herein again.
Operation S1020: Calculate an interval duration of the current test request; where interval durations corresponding to different test requests are not completely the same. In other words, interval durations corresponding to different at least two test requests are not completely the same.

To avoid that the hearing assistance receiving party easily finds a pattern and feeds back intuitively and consequently the hearing test result is inaccurate because the test audio is played periodically, the interval duration of the current test request may be calculated. The interval duration represents a time difference between a transmission time of the current test request and a transmission time of the next test request. In addition, interval durations corresponding to different test requests are not completely the same.

A specific manner of calculating the interval duration of the current test request may be flexibly set according to an actual requirement. In an exemplary embodiment, a random number may be generated within a specified range, and the random number is used as the interval duration of the current test request. That is, the interval duration of the test request is random. In another exemplary embodiment, multiple different interval durations may be preset, and an interval duration is sequentially extracted from the multiple interval durations in descending order (or in ascending order) as an interval duration of a test request. That is, the smallest interval duration is used as an interval duration of a first test request, and the second smallest interval duration is used as an interval duration of a second test request, and so on. After the longest interval duration is used as an interval duration of a test request, the smallest interval duration may be used as an interval duration of a subsequent test request, and so on.
Operation S1030: Calculate a transmission time of the next test request according to a transmission time and the interval duration of the current test request.

The transmission time of the next test request=the transmission time of the current test request+the interval duration of the current test request.
Operation S1040: Send the next test request to the assistance receiving terminal through the data transmission channel according to the transmission time of the next test request.

After the transmission time of the next test request is calculated, the next test request may be sent to the assistance receiving terminal through the data transmission channel according to the transmission time.

For specific implementation details of operation S210 to operation S230 shown in FIG. 10, refer to operation S210 to operation S230 shown in FIG. 2. For specific implementation details of operation S420 shown in FIG. 10, refer to operation S420 shown in FIG. 4. For specific implementation details of operation S820 shown in FIG. 10, refer to operation S820 shown in FIG. 8. Details are not described herein again.

In the embodiment shown in FIG. 10, transmission time intervals of adjacent test requests are not completely the same, to avoid that a user finds a pattern and feeds back intuitively and consequently the hearing test result is inaccurate because the test request is played periodically.

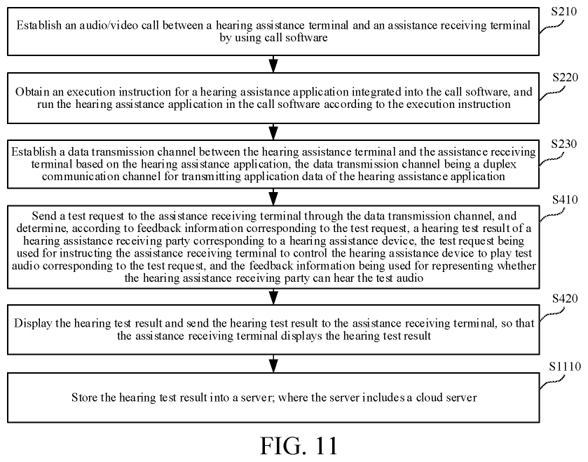

In an exemplary embodiment, FIG. 11 is a flowchart of a hearing assistance method based on FIG. 4. The method may be applied to the implementation environment shown in FIG. 1, and may be performed by the hearing assistance terminal 110 in the implementation environment shown in FIG. 1.

As shown in FIG. 11, the hearing assistance method may further include operation S1110. Details are described as follows:
Operation S1110: Store the hearing test result into a server; where the server includes a cloud server.

After obtaining the hearing test result, the hearing assistance terminal may store the hearing test result into the server. The server may be a cloud server, that is, the hearing test result is stored in cloud. Alternatively, the server may be a physical server.

For specific implementation details of operation S210 to operation S230 shown in FIG. 11, refer to operation S210 to operation S230 shown in FIG. 2. For specific implementation details of operation S410 and operation S420 shown in FIG. 11, refer to operation S410 and operation S420 shown in FIG. 4. Details are not described herein again.

In the embodiment shown in FIG. 11, the hearing test result is stored into the server, so that storage pressure on the hearing assistance terminal can be reduced, and the hearing assistance provider obtains the hearing test result from the server after logging into another terminal, thereby improving convenience.

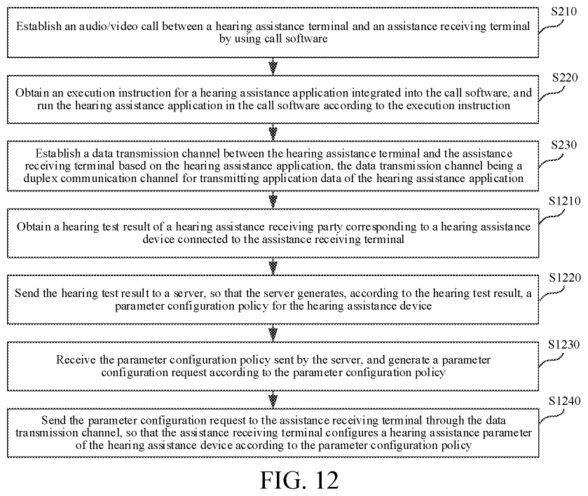

In an exemplary embodiment, FIG. 12 is a flowchart of a hearing assistance method based on FIG. 2. The method may be applied to the implementation environment shown in FIG. 1, and may be performed by the hearing assistance terminal 110 in the implementation environment shown in FIG. 1.

As shown in FIG. 12, operation S240 may include operation S1210 to operation S1240, and details are described as follows:
Operation S1210: Obtain a hearing test result of a hearing assistance receiving party corresponding to a hearing assistance device connected to the assistance receiving terminal.

The hearing assistance service includes a parameter configuration service configured for configuring a hearing assistance parameter of the hearing assistance device. The hearing assistance parameter of the hearing assistance device is configured to instruct the hearing assistance device how to help the hearing assistance receiving party improve hearing. Types of the hearing assistance parameter include, but are not limited to, a hearing assistance gain and a maximum power output (MPO). The hearing assistance gain is configured to instruct how to amplify sound. The MPO is configured for limiting a maximum sound intensity outputted by the hearing assistance device. The MPO cannot exceed an uncomfortable threshold of the hearing assistance receiving party.

To perform parameter configuration, the hearing test result of the hearing assistance receiving party may be obtained.

A specific manner of obtaining the hearing test result may be set flexibly according to an actual requirement. For example, in an exemplary embodiment, the hearing test result of the hearing assistance receiving party may be obtained through operation S410 and operation S420. Alternatively, in another exemplary embodiment, a historical hearing test result of the hearing assistance receiving party may be obtained from the server.
Operation S1220: Send the hearing test result to a server, so that the server generates, according to the hearing test result, a parameter configuration policy for the hearing assistance device.

To improve parameter configuration efficiency, the hearing test result is sent to the server, and the server generates a parameter configuration policy of the hearing assistance device according to the hearing test result. The parameter configuration policy is configured for configuring a hearing assistance parameter of the hearing assistance device, and the server may be a cloud server, or may be a physical server.

The parameter configuration policy includes hearing assistance parameters of the hearing assistance device at different sound frequencies, for example, an MPO and a hearing assistance gain of the hearing assistance device at different sound frequencies. In an embodiment, multiple sound intensity levels may be further set. Hearing assistance gains corresponding to a current sound frequency may include hearing assistance gains respectively corresponding to multiple sound intensity levels, so that for an input audio of different sound intensities corresponding to the same sound frequency, the hearing assistance device may amplify the input audio based on different hearing assistance gains and output the amplified input audio to the hearing assistance receiving party. A specific number of sound intensity levels and a sound intensity range corresponding to each sound intensity level may be set flexibly according to an actual requirement. For example, three sound intensity levels, namely, a high sound intensity level, a medium sound intensity level, and a low sound intensity level may be set. Sound intensity ranges respectively corresponding to the high sound intensity level, the medium sound intensity level, and the low sound intensity level are 70 to 80 db, 60 to 70 db, and 50 to 60 db. The sound intensity ranges respectively corresponding to the high sound intensity level, the medium sound intensity level, and the low sound intensity level may be properly adjusted to cover 75 db, 65 db, and 55 db. Hearing assistance gains of a sound frequency 1 KHz at the high sound intensity level, the medium sound intensity level, and the low sound intensity level are respectively 10 db, 12 db, and 15 db. If the hearing assistance device receives an audio whose sound frequency is 1 KHz and sound intensity is 55 db, the audio is amplified according to 15 db.

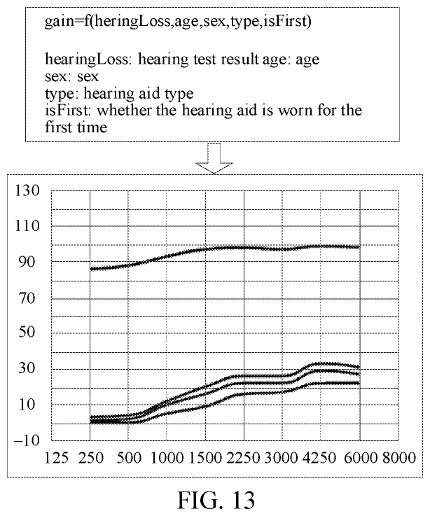

In an embodiment, in addition to the hearing test result, the parameter configuration policy may be generated based on an age, a gender, a type of the hearing assistance device, and whether the hearing assistance device is worn for the first time. For example, FIG. 13 is an exemplary calculation manner of the parameter configuration policy, where gain is a matching algorithm configured for generating the parameter configuration policy, the graph in lower half of FIG. 13 represents the parameter configuration policy, and 4 curves respectively represent an MPO and hearing assistance gains of the low sound intensity level, the medium sound intensity level, and the high sound intensity level from top to bottom.
Operation S1230: Receive the parameter configuration policy transmitted by the server, and generate a parameter configuration request according to the parameter configuration policy.

After generating the parameter configuration policy, the server sends the parameter configuration policy to the hearing assistance terminal. The hearing assistance terminal generates the parameter configuration request. The parameter configuration request is configured for requesting to configure a hearing assistance parameter of the hearing assistance device. The parameter configuration request includes the parameter configuration policy.
Operation S1240: Send the parameter configuration request to the assistance receiving terminal through the data transmission channel, so that the assistance receiving terminal configures a hearing assistance parameter of the hearing assistance device according to the parameter configuration policy.

After generating the parameter configuration request, the hearing assistance terminal may send the parameter configuration request to the assistance receiving terminal through the duplex communication connection to the assistance receiving terminal, and the assistance receiving terminal configures the hearing assistance parameter of the hearing assistance device according to the parameter configuration request.

For specific implementation details of operation S210 to operation S230 shown in FIG. 12, refer to operation S210 to operation S230 shown in FIG. 2. Details are not described herein again.

In an embodiment shown in FIG. 12, the server generates the parameter configuration policy, so as to improve a generation speed of the parameter configuration policy and improve a parameter configuration speed. In addition, calculation pressure of the hearing assistance terminal is reduced.

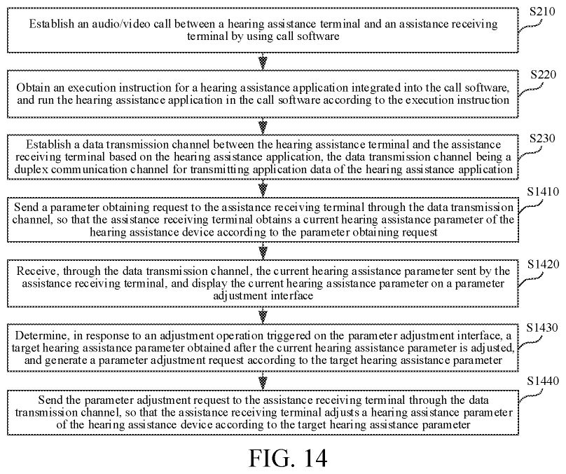

In an exemplary embodiment, FIG. 14 is a flowchart of a hearing assistance method based on FIG. 2. The method may be applied to the implementation environment shown in FIG. 1, and may be performed by the hearing assistance terminal 110 in the implementation environment shown in FIG. 1.

As shown in FIG. 14, operation S240 may include operation S1410 to operation S1440, and details are described as follows:
Operation S1410: Send a parameter obtaining request to the assistance receiving terminal through the data transmission channel, so that the assistance receiving terminal obtains a current hearing assistance parameter of the hearing assistance device according to the parameter obtaining request.

In this embodiment, the hearing assistance service includes a parameter adjustment service. To adjust the hearing assistance parameter of the hearing assistance device, the hearing assistance terminal may first generate the parameter obtaining request, and send the parameter obtaining request to the assistance receiving terminal through the data transmission channel. After receiving the parameter obtaining request, the assistance receiving terminal obtains the current hearing assistance parameter of the hearing assistance device according to the parameter obtaining request.
Operation S1420: Receive, through the data transmission channel, the current hearing assistance parameter transmitted by the assistance receiving terminal, and display the current hearing assistance parameter on a parameter adjustment interface.

After obtaining the current hearing assistance parameter of the hearing assistance device, the assistance receiving terminal sends the current hearing assistance parameter to the hearing assistance terminal through the data transmission channel; and the hearing assistance terminal displays the received current hearing assistance parameter on the parameter adjustment interface.
Operation S1430: Determine, in response to an adjustment operation triggered on the parameter adjustment interface, a target hearing assistance parameter obtained after the current hearing assistance parameter is adjusted, and generate a parameter adjustment request according to the target hearing assistance parameter.

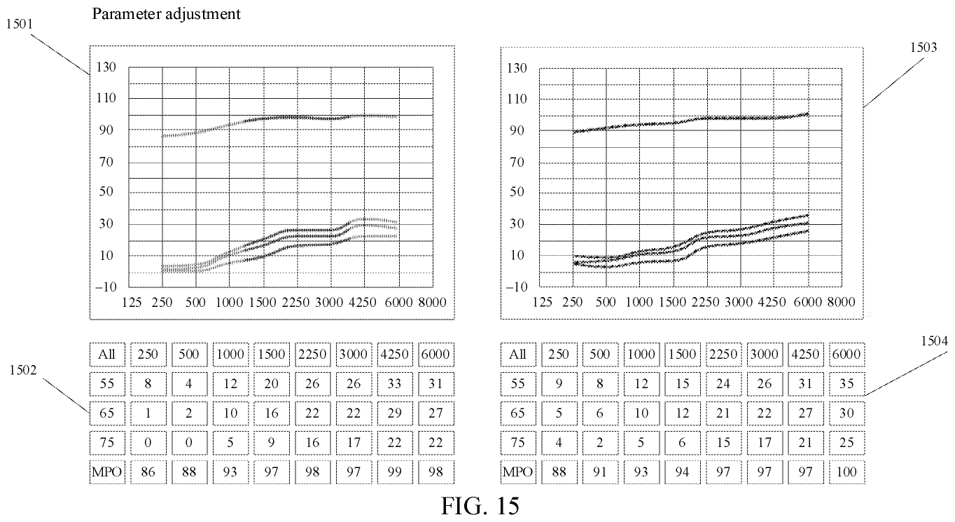

The hearing assistance provider may trigger an adjustment operation on the parameter adjustment interface, to adjust the current hearing assistance parameter, so that the hearing assistance terminal determines, according to the adjustment operation, the target hearing assistance parameter obtained after the current hearing assistance parameter is adjusted, and generates the parameter adjustment request including the target hearing assistance parameter. For example, as shown in FIG. 15, the parameter adjustment interface includes a current hearing assistance parameter. A graph 1501 and a table 1502 respectively represent hearing assistance parameters corresponding to the left ear. In the graph 1501, 4 curves respectively represent an MPO, a hearing assistance gain of a low sound intensity level, a hearing assistance gain of a medium sound intensity level, and a hearing assistance gain of a high sound intensity level corresponding to the left ear from top to bottom. A graph 1503 and a table 1504 respectively represent hearing assistance parameters corresponding to the right ear. In the graph 1503, 4 curves respectively represent an MPO, a hearing assistance gain of a low sound intensity level, a hearing assistance gain of a medium sound intensity level, and a hearing assistance gain of a high sound intensity level corresponding to the right ear from top to bottom. The hearing assistance provider may select and modify a corresponding hearing assistance parameter.

In an embodiment, the hearing assistance provider may obtain a related wearing status of the hearing assistance receiving party for the hearing assistance device based on an audio/video call with the hearing assistance receiving party or in another manner, so as to adjust the current hearing assistance parameter according to the wearing status.
Operation S1440: Send the parameter adjustment request to the assistance receiving terminal through the data transmission channel, so that the assistance receiving terminal adjusts a hearing assistance parameter of the hearing assistance device according to the target hearing assistance parameter.

After obtaining the parameter adjustment request, the hearing assistance terminal may send the parameter adjustment request to the assistance receiving terminal through the data transmission channel, and the assistance receiving terminal updates the hearing assistance parameter of the hearing assistance device according to the target hearing assistance parameter included in the parameter adjustment request.

For specific implementation details of operation S210 to operation S230 shown in FIG. 14, refer to operation S210 to operation S230 shown in FIG. 2. Details are not described herein again.

In the embodiment shown in FIG. 14, the hearing assistance parameter of the hearing assistance device may be adjusted through the data transmission channel and the audio/video call, thereby improving convenience of parameter adjustment. In addition, the hearing assistance parameter is adjusted based on the adjustment operation, thereby improving accuracy of parameter adjustment.

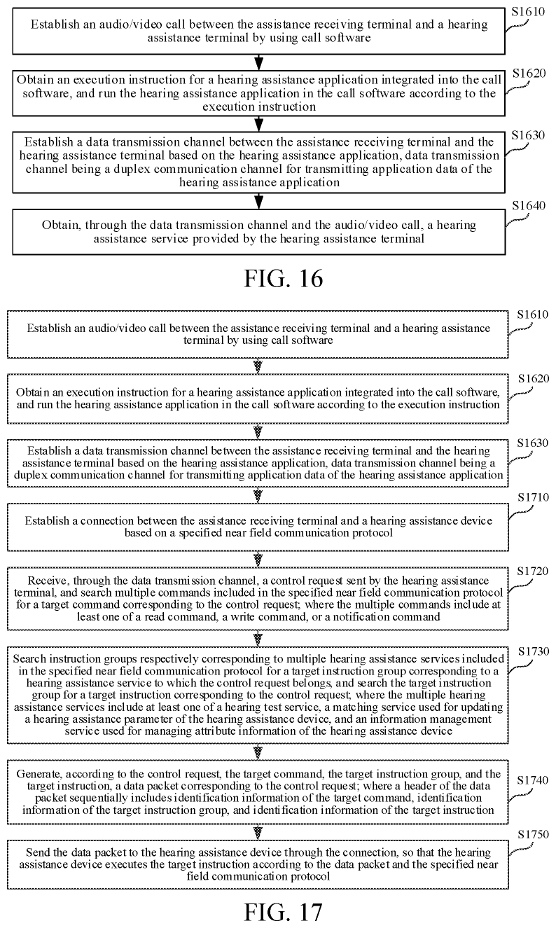

FIG. 16 is a flowchart of a hearing assistance method according to an exemplary embodiment of the present disclosure. The method may be applied to the implementation environment shown in FIG. 1, and may be performed by the assistance receiving terminal 120 in the implementation environment shown in FIG. 1.

As shown in FIG. 16, in an exemplary embodiment, the hearing assistance method may include operation S1610 to operation S1640, which are described in detail as follows:
Operation S1610: Establish an audio/video call between the assistance receiving terminal and a hearing assistance terminal by using call software.

The call software is installed in the assistance receiving terminal, and an audio/video call may be established between the assistance receiving terminal and the hearing assistance terminal by using the call software. For a specific process in which the assistance receiving terminal establishes the audio/video call with the hearing assistance terminal, refer to the foregoing description, and details are not described herein again.
Operation S1620: Obtain an execution instruction for a hearing assistance application integrated into the call software, and run the hearing assistance application in the call software according to the execution instruction.

In an embodiment, the execution instruction may be triggered by the hearing assistance receiving party. Alternatively, if receiving an invitation request from the hearing assistance terminal, the assistance receiving terminal may automatically generate the execution instruction.
Operation S1630: Establish a data transmission channel between the assistance receiving terminal and the hearing assistance terminal based on the hearing assistance application, data transmission channel being a duplex communication channel for transmitting application data of the hearing assistance application.

For a specific process of establishing the data transmission channel based on the hearing assistance application, refer to the description in the foregoing embodiment, and details are not described herein again.
Operation S1640: Obtain, through the data transmission channel and the audio/video call, a hearing assistance service provided by the hearing assistance terminal.

After the audio/video call and the data transmission channel are established, the hearing assistance receiving party and the hearing assistance provider may communicate with each other by using the audio/video call. Meanwhile, the application data of the hearing assistance application is transmitted between the hearing assistance terminal and the assistance receiving terminal through the data transmission channel, to transmit data triggered by the hearing assistance provider or the hearing assistance receiving party in the hearing assistance application to the other party. Therefore, this implements a remote service of the hearing assistance application, and the hearing assistance service is received through the assistance receiving terminal.

In the embodiment shown in FIG. 16, the audio/video call implements communication between the hearing assistance provider corresponding to the hearing assistance terminal and the hearing assistance receiving party corresponding to the assistance receiving terminal, thereby improving convenience and accuracy of the hearing assistance service. In addition, the audio/video call is established based on the call software, so that stability of the audio/video call can be improved. The hearing assistance application does not need to integrate an audio/video call function, and the hearing assistance application is hosted in the call software and runs based on the call software. This can reduce difficulty in developing the hearing assistance application, improve stability of the hearing assistance application, and improve quality of the hearing assistance service.

In an exemplary embodiment, FIG. 17 is a flowchart of a hearing assistance method based on FIG. 16. The method may be applied to the implementation environment shown in FIG. 1, and may be performed by the assistance receiving terminal 120 in the implementation environment shown in FIG. 1.

As shown in FIG. 17, operation S1640 may include operation S1710 to operation S1750, and details are described as follows:
Operation S1710: Establish a connection between the assistance receiving terminal and a hearing assistance device based on a target near field communication protocol.

In a process of providing a hearing assistance service, participation of the hearing assistance device is usually needed. Therefore, the connection between the assistance receiving terminal and the hearing assistance device needs to be established.

To improve quality of the hearing assistance service, a near field communication protocol configured for the hearing assistance service may be preset, and the assistance receiving terminal establishes the connection between the assistance receiving terminal and the hearing assistance device based on the target near field communication protocol. The near field communication protocol is a protocol configured for establishing a connection and transmitting data between the assistance receiving terminal and the hearing assistance device, and types of the protocol include, but are not limited to, Bluetooth and the like.

It is specified that the type of the near field communication protocol includes, but is not limited to, Bluetooth and the like.
Operation S1720: Receive, through the data transmission channel, a control request transmitted by the hearing assistance terminal, and search multiple commands included in the target near field communication protocol for a target command corresponding to the control request; where the multiple commands include at least one of a read command, a write command, or a notification command.

To provide the hearing assistance service, the hearing assistance terminal may generate the control request, for example, the parameter configuration request, the parameter adjustment request, and the test request. Then, the control request is sent to the assistance receiving terminal through the data transmission channel.

After receiving the control request, the assistance receiving terminal needs to control the hearing assistance device according to the control request. To control the hearing assistance device, the assistance receiving terminal needs to generate, based on the target near field communication protocol, a data packet corresponding to the control request. The target near field communication protocol includes multiple commands. The multiple commands include a read command, a write command, and a notification command. The read command is configured to instruct to read data from the hearing assistance device, the write command is configured to instruct to write data into the hearing assistance device, and the notification command is configured to instruct the hearing assistance device to perform notification. The assistance receiving terminal may select the target command corresponding to the control request from the multiple commands. If the control request is configured to instruct to read data from the hearing assistance device, the target command is a read command. For example, the parameter obtaining request is configured for obtaining a hearing assistance parameter of the hearing assistance device, and corresponds to the read command. For another example, the parameter configuration request is configured for configuring a hearing assistance parameter of the hearing assistance device, and corresponds to a write command.
Operation S1730: Search instruction groups respectively corresponding to multiple hearing assistance services included in the target near field communication protocol for a target instruction group corresponding to a hearing assistance service to which the control request belongs, and search the target instruction group for a target instruction corresponding to the control request; where the multiple hearing assistance services include at least one of a hearing test service, a matching service configured to update a hearing assistance parameter of the hearing assistance device, and an information management service configured to manage attribute information of the hearing assistance device.

In the near field communication protocol, corresponding instruction groups are set for different hearing assistance services. The instruction groups include instructions that need to be used by corresponding hearing assistance services. The hearing assistance services include, but are not limited to, at least one of a hearing test service, a matching service configured to update a hearing assistance parameter of a hearing assistance device, an information management service configured to manage attribute information of the hearing assistance device, and the like. The matching service includes a parameter configuration service, a parameter adjustment service, and the like.

The instruction groups respectively corresponding to the multiple hearing assistance services included in the target near field communication protocol may be searched for the target instruction group corresponding to the hearing assistance service to which the control request belongs, and the target instruction group may be searched for the target instruction corresponding to the control request. For example, if the control request is a playing request corresponding to the hearing test service, the target instruction group is an instruction group corresponding to the hearing test service, and the target instruction is a playing instruction.

The target instruction group is a corresponding instruction group found by the assistance receiving terminal for the hearing assistance service to which the control request belongs, from the instruction groups respectively corresponding to the multiple hearing assistance services included in the target near field communication protocol. The instruction group includes an instruction that needs to be used by a corresponding hearing assistance service. The target instruction is a corresponding instruction found by the assistance receiving terminal for the control request from the target instruction group, and the hearing assistance device performs a corresponding operation according to the instruction.
Operation S1740: Generate, according to the control request, the target command, the target instruction group, and the target instruction, a data packet corresponding to the control request; where a header of the data packet sequentially includes identification information of the target command, identification information of the target instruction group, and identification information of the target instruction.

The assistance receiving terminal may generate, according to the control request, the target command, the target instruction group, and the target instruction, the data packet corresponding to the control request, so as to obtain a data packet matching the target near field communication protocol.

To enable the hearing assistance device to quickly know the purpose of the data packet, the header of the data packet corresponding to the control request sequentially includes the identification information of the target command, the identification information of the target instruction group, and the identification information of the target instruction. The identification information may be an identification number.

In an exemplary embodiment, the header of the data packet transmitted between the assistance receiving terminal and the hearing assistance device sequentially includes an application layer protocol version, an information signal, a command identification number, a transmission direction, an instruction group identification number, an instruction identification number, and a payload. The transmission direction is configured for indicating whether the data packet is transmitted from the assistance receiving terminal to the hearing assistance device, or is transmitted from the hearing assistance device to the assistance receiving terminal.
Operation S1750: Send the data packet to the hearing assistance device through the connection, so that the hearing assistance device executes the target instruction according to the data packet and the target near field communication protocol.

The assistance receiving terminal sends the data packet to the hearing assistance device. The hearing assistance device may parse the received data packet according to the target near field communication protocol, so as to execute the target instruction corresponding to the received data packet.

In an embodiment, the data packet sent by the hearing assistance device to the assistance receiving terminal may also be generated in the foregoing manner.

For specific implementation details of operation S1610 to operation S1630 shown in FIG. 17, refer to operation S1610 to operation S1630 shown in FIG. 16. Details are not described herein again.

In the embodiment shown in FIG. 17, the assistance receiving terminal and the hearing assistance device perform data transmission based on the target near field communication protocol, so as to obtain the hearing assistance service provided by the hearing assistance application. In this way, as long as the hearing assistance device supports the target near field communication protocol, the hearing assistance service provided by the hearing assistance application can be obtained.

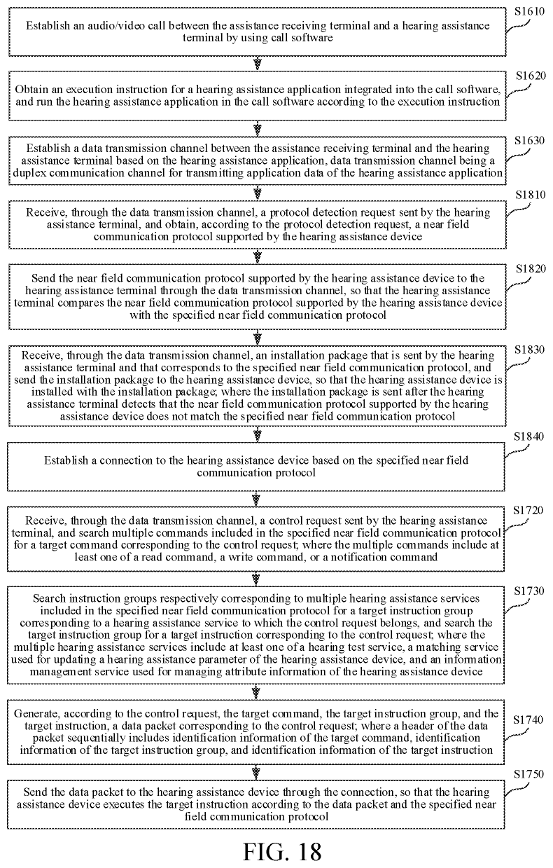

In an exemplary embodiment, FIG. 18 is a flowchart of a hearing assistance method based on FIG. 17. The method may be applied to the implementation environment shown in FIG. 1, and may be performed by the assistance receiving terminal 120 in the implementation environment shown in FIG. 1.

As shown in FIG. 18, operation S1710 may include operation S1810 to operation S1840, and details are described as follows:
Operation S1810: Receive, through the data transmission channel, a protocol detection request transmitted by the hearing assistance terminal, and obtain, according to the protocol detection request, a near field communication protocol supported by the hearing assistance device.

To avoid that a hearing assistance service cannot be provided to the hearing assistance device because a near field communication protocol supported by the hearing assistance device does not match a target near field communication protocol, the hearing assistance terminal may generate the protocol detection request, to detect the near field communication protocol supported by the hearing assistance device, and send the protocol detection request to the assistance receiving terminal through the data transmission channel.

After receiving the protocol detection request, the assistance receiving terminal obtains, according to the protocol detection request, the near field communication protocol supported by the hearing assistance device.

In an embodiment, the assistance receiving terminal may establish a connection to the hearing assistance device based on the near field communication protocol supported by the hearing assistance device, so as to perform communication with the hearing assistance device through the connection, to obtain the near field communication protocol supported by the hearing assistance device.
Operation S1820: Send the near field communication protocol supported by the hearing assistance device to the hearing assistance terminal through the data transmission channel, so that the hearing assistance terminal compares the near field communication protocol supported by the hearing assistance device with the target near field communication protocol.

The assistance receiving terminal sends the near field communication protocol supported by the hearing assistance device to the hearing assistance terminal through the data transmission channel, and the hearing assistance terminal compares the near field communication protocol supported by the hearing assistance device with the target near field communication protocol, so as to detect whether the near field communication protocol supported by the hearing assistance device matches the target near field communication protocol.
Operation S1830: Receive, through the data transmission channel, an installation package that is transmitted by the hearing assistance terminal and that corresponds to the target near field communication protocol, and send the installation package to the hearing assistance device, so that the hearing assistance device is installed with the installation package; where the installation package is transmitted after the hearing assistance terminal detects that the near field communication protocol supported by the hearing assistance device does not match the target near field communication protocol.

If detecting that the near field communication protocol supported by the hearing assistance device does not match the target near field communication protocol, the hearing assistance terminal sends the installation package corresponding to the target near field communication protocol to the assistance receiving terminal through the data transmission channel. The assistance receiving terminal sends the installation package to the hearing assistance device. The hearing assistance device performs installation after receiving the installation package, so that the hearing assistance device supports the target near field communication protocol.

If detecting that the near field communication protocol supported by the hearing assistance device matches the target near field communication protocol, the hearing assistance terminal may send a connection establishment request to the assistance receiving terminal through the data transmission channel, so that the assistance receiving terminal establishes a connection between the assistance receiving terminal and the hearing assistance device according to the connection establishment request based on the target near field communication protocol.
Operation S1840: Establish a connection to the hearing assistance device based on the target near field communication protocol.

After the installation package corresponding to the target near field communication protocol is installed on the hearing assistance device, the hearing assistance device supports the target near field communication protocol, and the assistance receiving terminal may establish a connection to the hearing assistance device according to the target near field communication protocol.

For specific implementation details of operation S1610 to operation S1630 shown in FIG. 18, refer to operation S1610 to operation S1630 shown in FIG. 16. For specific implementation details of operation S1720 and operation S1750 shown in FIG. 18, refer to operation S1720 and operation S1750 shown in FIG. 17. Details are not described herein again.

In the embodiment shown in FIG. 18, it may be detected whether the near field communication protocol supported by the hearing assistance device matches the target near field communication protocol, and if not, the installation package corresponding to the target near field communication protocol may be sent to the hearing assistance device for installation, so that the hearing assistance device supports the target near field communication protocol, to improve a hearing assistance service and improve a success rate of parameter configuration and the like.

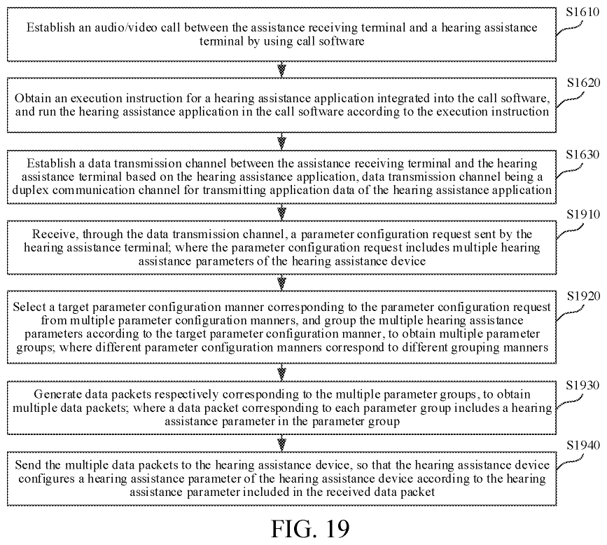

In an exemplary embodiment, FIG. 19 is a flowchart of a hearing assistance method based on FIG. 16. The method may be applied to the implementation environment shown in FIG. 1, and may be performed by the assistance receiving terminal 120 in the implementation environment shown in FIG. 1.

As shown in FIG. 19, operation S1640 may include operation S1910 to operation S1940, and details are described as follows:
Operation S1910: Receive, through the data transmission channel, a parameter configuration request transmitted by the hearing assistance terminal; where the parameter configuration request includes multiple hearing assistance parameters of the hearing assistance device.

The parameter configuration request is configured for configuring a hearing assistance parameter of the hearing assistance device, and may include multiple hearing assistance parameters of the hearing assistance device, for example, include a hearing assistance gain and an MPO of the hearing assistance device at different sound frequencies.
Operation S1920: Select a target parameter configuration manner corresponding to the parameter configuration request from multiple parameter configuration manners, and group the multiple hearing assistance parameters according to the target parameter configuration manner, to obtain multiple parameter groups; where different parameter configuration manners correspond to different grouping manners.

In this embodiment, different parameter configuration manners are set, and different parameter configuration manners correspond to different grouping manners of the multiple hearing assistance parameters included in the parameter configuration request. For example, hearing at the left ear and hearing at the right ear are different, and hearing assistance parameters corresponding to a left channel and a right channel are different. Therefore, in a parameter configuration manner, hearing assistance parameters that belong to the same channel may be grouped into one group according to channels to which the hearing assistance parameters belong. In another exemplary parameter configuration manner, grouping may be performed according to channels and sound frequencies to which hearing assistance parameters belong, so as to group hearing assistance parameters that belong to the same ear and the same sound frequency into one group.

The target parameter configuration manner is a manner selected from the multiple parameter configuration manners and configured for configuring the parameter configuration request. A specific process of selecting the target parameter configuration manner corresponding to the parameter configuration request from the multiple parameter configuration manners may be flexibly set according to an actual requirement. In an exemplary embodiment, different versions of near field communication protocols used to create the connection between the hearing assistance device and the assistance receiving terminal correspond to different maximum transmission units (MTU). Therefore, the target parameter configuration manner may be selected according to the version of the near field communication protocol corresponding to the hearing assistance device and the assistance receiving terminal. As the near field communication protocol corresponds to a larger MTU, a target parameter configuration manner in which a parameter group after grouping includes a higher data volume of hearing assistance parameters may be selected. For example, if the MTU is relatively large, a parameter configuration manner in which grouping is performed according to channels to which hearing assistance parameters belong may be selected. If the MTU is relatively small, a parameter configuration manner in which grouping is performed according to channels and sound frequencies to which hearing assistance parameters belong may be selected.
Operation S1930: Generate data packets respectively corresponding to the multiple parameter groups, to obtain multiple data packets; where a data packet corresponding to each parameter group includes a hearing assistance parameter in the parameter group.

In a process of generating a data packet corresponding to a parameter group, a hearing assistance parameter included in the parameter group is added to the data packet.
Operation S1940: Send the multiple data packets to the hearing assistance device, so that the hearing assistance device configures a hearing assistance parameter of the hearing assistance device according to the hearing assistance parameter included in the received data packet.

After the data packets respectively corresponding to the multiple parameter groups are obtained, that is, the multiple data packets are obtained, the assistance receiving terminal may send the multiple data packets to the hearing assistance device, so that the hearing assistance device configures a hearing assistance parameter of the hearing assistance device according to the hearing assistance parameter included in the received data packet.

In an embodiment, in a process of transmitting the multiple data packets to the hearing assistance device, the current data packet may be first sent to the hearing assistance device, so that the hearing assistance device updates a hearing assistance parameter of the device according to a hearing assistance parameter included in the current data packet. If the update succeeds, an update success notification is sent to the assistance receiving terminal, so that the assistance receiving terminal sends a next data packet to the hearing assistance device according to the update success notification, until the hearing assistance parameters included in the multiple parameter groups are all successfully updated. In an embodiment, if the update fails, a data packet on which the failed update is based may be retransmitted.

In a parameter adjustment process, parameter adjustment may also be performed in a manner of operation S1920 to operation S1940. For specific implementation details of operation S1610 to operation S1630 shown in FIG. 19, refer to operation S1610 to operation S1630 shown in FIG. 16, and details are not described herein again.

In the embodiment shown in FIG. 19, the hearing assistance parameter of the hearing assistance device may be configured according to different parameter configuration manners, thereby improving parameter configuration flexibility.

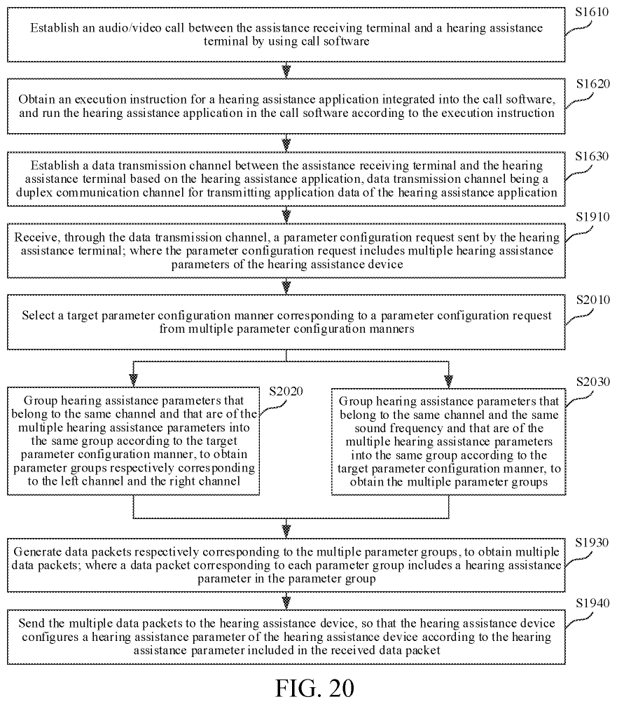

In an exemplary embodiment, FIG. 20 is a flowchart of a hearing assistance method based on FIG. 19. The method may be applied to the implementation environment shown in FIG. 1, and may be performed by the assistance receiving terminal 120 in the implementation environment shown in FIG. 1.

As shown in FIG. 20, when the multiple hearing assistance parameters include hearing assistance parameters respectively corresponding to a left channel and a right channel, and a hearing assistance parameter corresponding to each channel includes hearing assistance parameters respectively corresponding to multiple sound frequencies, operation S1920 may include operation S2010 to operation S2030, and details are described as follows:
Operation S2010: Select a target parameter configuration manner corresponding to a parameter configuration request from multiple parameter configuration manners.

A specific selection manner may be set flexibly according to an actual requirement.
Operation S2020: Group hearing assistance parameters that belong to the same channel and that are of the multiple hearing assistance parameters into the same group according to the target parameter configuration manner, to obtain parameter groups respectively corresponding to the left channel and the right channel.

Hearing statuses of the left ear and the right ear of the hearing assistance receiving party are different. Therefore, the multiple hearing assistance parameters include hearing assistance parameters respectively corresponding to the left channel and the right channel. The hearing assistance parameter corresponding to the left channel is configured for helping the left ear, and the hearing assistance parameter corresponding to the right channel is configured for helping the right ear. A hearing assistance parameter corresponding to each channel includes hearing assistance parameters of the channel at multiple sound frequencies.

When grouping the hearing assistance parameters, hearing assistance parameters that belong to the same channel may be grouped into the same group, so as to obtain parameter groups respectively corresponding to the left channel and the right channel. That is, the hearing assistance parameter is sent to the hearing assistance device in unit of a channel.
Operation S2030: Group hearing assistance parameters that belong to the same channel and the same sound frequency and that are of the multiple hearing assistance parameters into the same group according to the target parameter configuration manner, to obtain the multiple parameter groups.

When grouping the hearing assistance parameters, hearing assistance parameters that belong to the same channel and the same sound frequency and that are of the multiple hearing assistance parameters may also be grouped into the same group, to obtain the multiple parameter groups. That is, the hearing assistance parameter is sent to the hearing assistance device in unit of a single channel and a single sound frequency.

For specific implementation details of operation S1610 to operation S1630 shown in FIG. 20, refer to operation S1610 to operation S1630 shown in FIG. 16. For specific implementation details of operation S1910, operation S1930, and operation S1940 shown in FIG. 20, refer to operation S1910, operation S1930, and operation S1940 shown in FIG. 19. Details are not described herein again.

In the embodiment shown in FIG. 20, hearing assistance parameters that belong to the same channel may be grouped into the same group, or hearing assistance parameters that belong to the same channel and the same sound frequency may be grouped into the same group, thereby improving the flexibility of parameter grouping and improving the flexibility of parameter configuration.

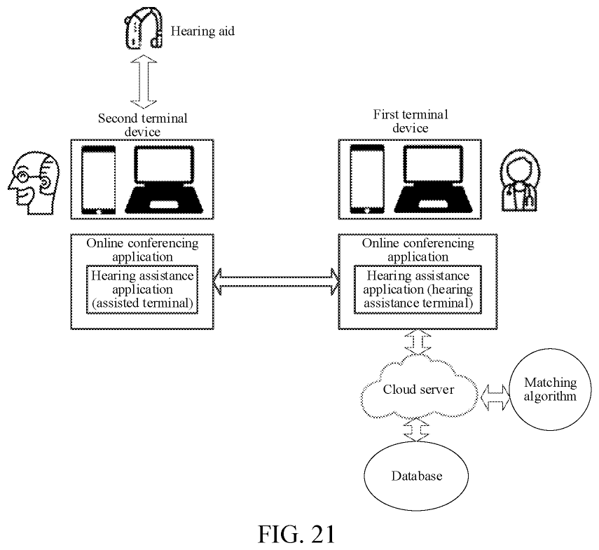

To better understand the present disclosure, an example in which a hearing assistance provider is an audiologist, and a hearing assistance receiving party is a hearing impaired user is configured for description herein. FIG. 21 is a schematic diagram of an implementation environment according to the present disclosure. The implementation environment includes a first terminal device and a second terminal device. The first terminal device is a terminal device used by a user, and the second terminal device is a terminal device used by an audiologist. An online conferencing application is installed in both the first terminal device and the second terminal device. A hearing assistance application in a user mode runs in the online conferencing application of the first terminal device, and a hearing assistance application in an audiologist mode runs in the online conferencing application of the second terminal device. A point-to-point data transmission channel is established between the first terminal device and the second terminal device. The first terminal device is connected, based on a Bluetooth low energy (BLE) protocol, to a hearing assistance device worn by a user. The second terminal device is connected to a cloud server, and the cloud server includes a cloud database and a matching algorithm.

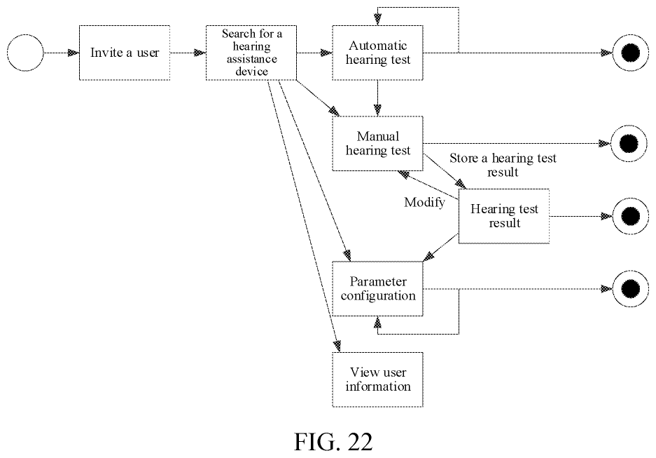

Referring to FIG. 22, a process of performing the hearing assistance service includes the following processes 1.1 to 1.7:
Process 1.1: Invite a user.

The audiologist and the user may agree on a matching time, and reserve an online conference in the online conferencing application. Then, the audiologist may log into the online conferencing application on the second terminal device, and join the reserved online conference. The user may log into the online conferencing application on the first terminal device, and join the reserved online conference, thereby establishing an audio/video call between the audiologist and the user.

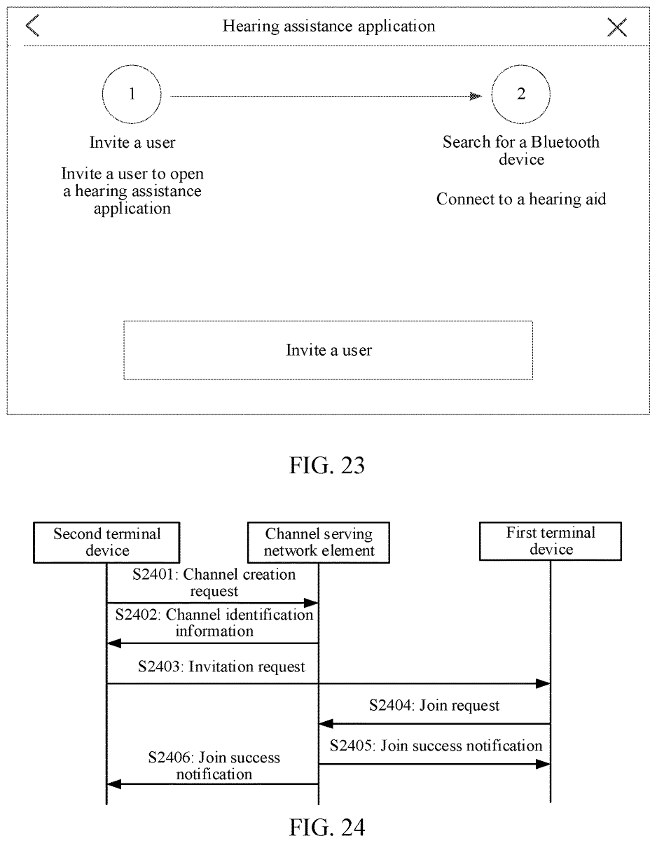

The audiologist searches for a hearing assistance application in the online conferencing application, opens the hearing assistance application, selects the audiologist mode, and then initiates an invitation request to the user. For example, FIG. 23 is an exemplary user invitation interface. The audiologist may trigger an invitation request by using a control “invite a user”, and send the invitation request to the first terminal device. The first terminal device displays the invitation. If the user triggers to agree to the invitation, a point-to-point data transmission channel between the hearing assistance application in the first terminal device and the hearing assistance application in the second terminal device is created based on a point-to-point communication protocol, so as to implement remote transfer of application data of the hearing assistance application through the channel. Referring to FIG. 24, a process of creating the point-to-point data transmission channel includes operation S2401 to operation S2406. Details are described as follows:
Operation S2401: A second terminal device sends a channel creation request to a channel serving network element.

The channel serving network element is configured to provide a channel management function. After the audiologist triggers the control “invite a user”, the second terminal device may generate the channel creation request. The channel creation request includes identification information of the audiologist in the hearing assistance application, for example, an account of the audiologist.
Operation S2402: The channel serving network element sends channel identification information to the second terminal device.

The channel serving network element generates channel identification information according to the channel creation request, records a relationship between the channel identification information and the identification information of the audiologist, and sends the channel identification information to the second terminal device.
Operation S2403: The second terminal device sends an invitation request to a first terminal device.

The invitation request includes channel identification information.
Operation S2404: The first terminal device sends a join request to the channel serving network element.

The join request includes the channel identification information.
Operation S2405: The channel serving network element sends a join success notification to the first terminal device.

After receiving the join request, the channel serving network element stores association among identification information of the user (for example, an account of the user in the hearing assistance application), the identification information of the audiologist, and the channel identification information, so as to create the point-to-point data transmission channel between the first terminal device and the second terminal device, and feed back the join success notification to the first terminal device.
Operation S2406: The channel serving network element sends the join success notification to the second terminal device.

The channel serving network element sends the join success notification to the second terminal device, so that the second terminal device determines that the point-to-point data transmission channel is successfully created. In a subsequent process, the channel serving network element may forward data between the first terminal device and the second terminal device. The channel serving network element may find the corresponding point-to-point data transmission channel according to the identification information of the audiologist and the user and forward data.
Process 1.2: Search for a hearing assistance device.

After the point-to-point data transmission channel between the first terminal device and the second terminal device is created, the second terminal device may notify the first terminal device to search for a hearing assistance device, and a connection is established between the assistance receiving terminal and the hearing assistance device based on BLE.

In an embodiment, the audiologist may instruct, by using an audio/video call, the user to turn on the hearing assistance device and to search for the hearing assistance device by using the first terminal device.
Process 1.3: Automatic hearing test.

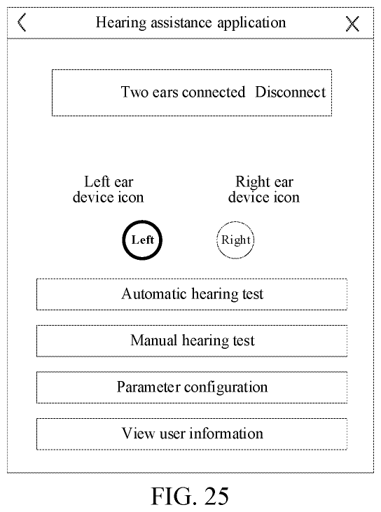

After the hearing assistance device is connected, a service selection interface may be displayed on the second terminal device. Referring to FIG. 25, the service selection interface includes automatic hearing test, manual hearing test, parameter configuration, and user information viewing. The audiologist may select a service to be performed.

If the audiologist selects the automatic hearing test service, a hearing test may be automatically performed on the user.
Process 1.4: Manual hearing test.

If the audiologist selects manual hearing test, a hearing test may be manually performed on the user.

In an embodiment, regardless of whether manual hearing test or automatic hearing test is performed, before the hearing test, the audiologist determines, by using an audio/video call, whether the user wears the hearing assistance device as required, and whether ambient noise is suitable, and if yes, the hearing test is performed.

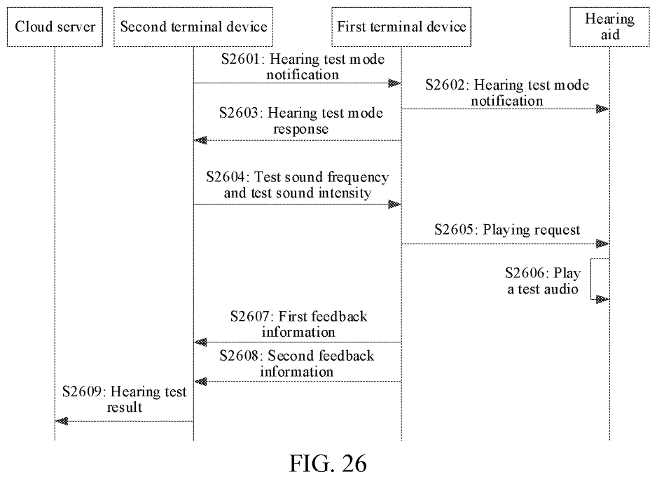

In a hearing test process, referring to FIG. 26, the hearing test process includes operation S2601 to operation S2609. Details are described as follows:
Operation S2601: The second terminal device sends a notification of a hearing test mode to the first terminal device.

The second terminal device sends the notification of the hearing test mode to the first terminal device, so that the first terminal device enters the test mode.
Operation S2602: The first terminal device sends the notification of the hearing test mode to the hearing assistance device.

The first terminal device sends the notification of the hearing test mode to the hearing assistance device, so that the hearing assistance device enters the hearing test mode.
Operation S2603: The first terminal device sends a response of entering the hearing test mode to the second terminal device.
Operation S2604: The second terminal device sends a test sound frequency and a test sound intensity to the first terminal device.

In an exemplary embodiment, a value range of the test sound frequency may be (500 Hz, 1 kHz, 1500 Hz, 2 kHz, 3 kHz, 4 kHz, 6 kHz, and 8 kHz), and a value range of the test sound intensity may be (0, 5, 10, 15, 20, . . . , 115, 120), that is, discrete values from 0 db to 120 db with the interval of 5 db. The second terminal device sends a test sound frequency and a test sound intensity to the first terminal device each time, and then sends a next test sound frequency and a next test sound intensity after obtaining corresponding feedback information.

In the case of manual hearing test, the test sound frequency and the test sound intensity may be set by the audiologist.

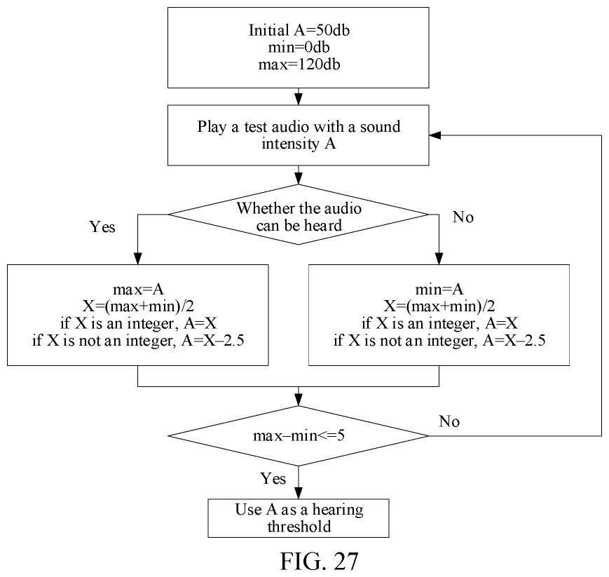

In the case of automatic audio test, referring to FIG. 27, a hearing threshold of a user at a current test sound frequency may be determined by using a method shown in FIG. 27: Initial A, initial min, and initial max are obtained, where A is a test sound intensity, min is a minimum sound intensity, and max is a maximum sound intensity. In FIG. 27, for example, initial A=50 db, initial min=0 db, and initial max=120 db. Then, A and the current test sound frequency are sent to the first terminal device, so as to control the hearing assistance device to play a test audio whose sound intensity is A. Then, it is determined, according to feedback information of the user, whether the user can hear the test audio. If yes, it is set that max=A, and then A is updated according to an average value of max and min. To ensure that A is an integer of 5, it is first set that X=(max+min)÷2. Then, if X is an integer, A=X, and if X is not an integer, A=X−2.5. If not, it is set that min=A, and then A is updated according to the average value of max and min. To ensure that A is an integer of 5, it is first set that X=(max+min)÷2. Then, if X is an integer, A=X, and if X is not an integer, A=X−2.5. After A, max, and min are updated, it is determined whether (max-min) is less than or equal to a specified intensity threshold. Herein, for example, the intensity threshold is 5. If yes, A is used as a hearing threshold. If not, return to the operation of playing the test audio whose sound intensity is A and repeat.
Operation S2605: The first terminal device sends a playing request to the hearing assistance device.

The playing request includes a test sound frequency and a test sound intensity.
Operation S2606: The hearing assistance device plays a test audio.

The test audio matches the test sound frequency and the test sound intensity.
Operation S2607: The first terminal device sends, to the second terminal device, first feedback information representing that the test audio can be heard.

The user may input, by using the first terminal device, feedback information indicating that the test audio can be heard, and the first terminal device sends the feedback information to the second terminal device.
Operation S2608: The first terminal device sends, to the second terminal device, second feedback information representing that the test audio cannot be heard.

After sending the playing request to the second terminal device, the first terminal device sets a timer for countdown based on a specified duration. If the user does not feed back before the timer ends, feedback information representing that the test audio cannot be heard is sent to the second terminal device.
Operation S2604 to operation S2608 are performed for multiple times, until a hearing test result is obtained.
Operation S2609: The second terminal device sends a hearing test result to a cloud server.

The cloud server stores the hearing test result.

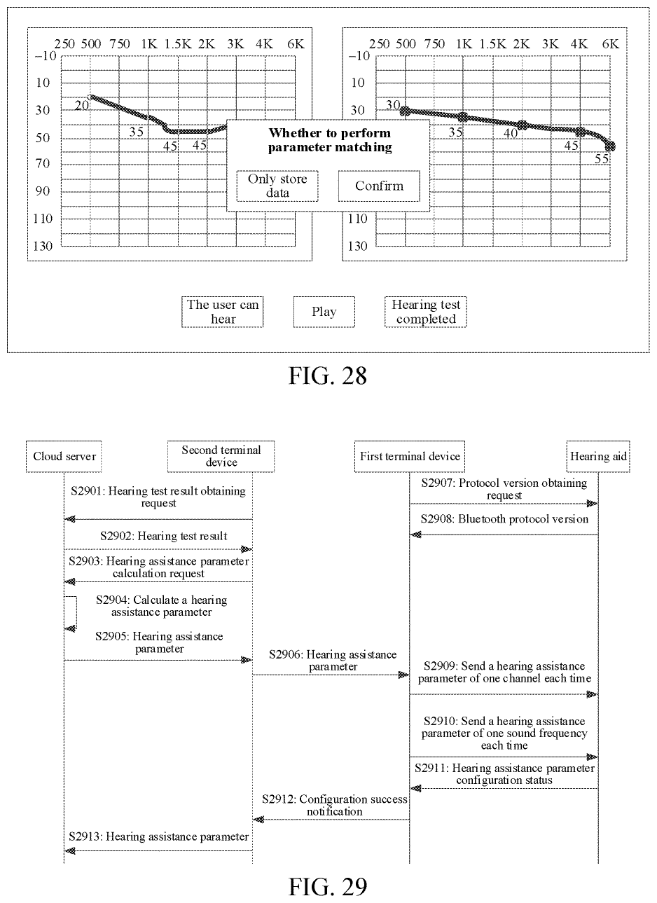

After the hearing test result is obtained through manual hearing test, the hearing test result may be stored, or modification is performed. Alternatively, FIG. 28 is a selection interface after the hearing test result is obtained, and storing the hearing test result may be selected, or entering parameter matching is confirmed.
Process 1.5: Hearing test result.

The hearing test result may be obtained through manual hearing test and automatic hearing test.
Process 1.6: Parameter matching.

Parameter matching includes parameter configuration and parameter modification.

Referring to FIG. 29, the parameter configuration includes operation S2901 to operation S2913. Details are described as follows:
Operation S2901: A second terminal device sends a hearing test result obtaining request to a cloud server.
Operation S2902: The cloud server sends a hearing test result to the second terminal device.
Operation S2903: The second terminal device sends a hearing assistance parameter calculation request to the cloud server.

The hearing assistance parameter calculation request includes a hearing test result.
Operation S2904: The cloud server calculates a hearing assistance parameter.

The hearing assistance parameter includes a [3*F] wdrc matrix and a [1*F] MPO matrix, where F represents a sound frequency, [3*F] wdrc includes hearing assistance gains respectively corresponding to the high sound intensity level, the medium sound intensity level, and the low sound intensity level at different sound frequencies F, and the [1*F] MPO matrix includes MPOs at different sound frequencies.
Operation S2905: The cloud server sends the hearing assistance parameter to the second terminal device.
Operation S2906: The second terminal device sends the hearing assistance parameter to the first terminal device.
Operation S2907: The first terminal device sends a protocol version obtaining request to the hearing assistance device.
Operation S2908: The hearing assistance device sends a Bluetooth protocol version to the first terminal device.

The hearing assistance device sends the Bluetooth protocol version of the hearing assistance device to the first terminal device according to the protocol version obtaining request.
Operation S2909: The first terminal device sends a hearing assistance parameter of one channel to the hearing assistance device each time.

The first terminal device may determine a hearing assistance parameter sending mode according to the Bluetooth protocol version. If an MTU supported by the Bluetooth protocol version is relatively large, the first terminal device may send a hearing assistance parameter of one channel each time, until all hearing assistance parameters are sent to the hearing assistance device.
Operation S2910: The first terminal device sends a hearing assistance parameter of one sound frequency to the hearing assistance device each time.

If the MTU supported by the Bluetooth protocol version is relatively small, the first terminal device may send a hearing assistance parameter corresponding to one sound frequency each time, until all hearing assistance parameters are sent to the hearing assistance device. The hearing assistance parameter sent each time may be encapsulated into one data packet for sending.
Operation S2911: The hearing assistance device sends a hearing assistance parameter configuration status to the first terminal device.
Operation S2912: The second terminal device sends a configuration success notification to the first terminal device.

The configuration success notification is generated after the hearing assistance parameter configuration status indicates successful configuration.
Operation S2913: The second terminal device stores the hearing assistance parameter in a cloud server.

In the cloud server, hearing assistance parameters may be classified based on manufacturers of hearing assistance parameters, so that a manufacturer can conveniently obtain a hearing assistance parameter from the cloud server.

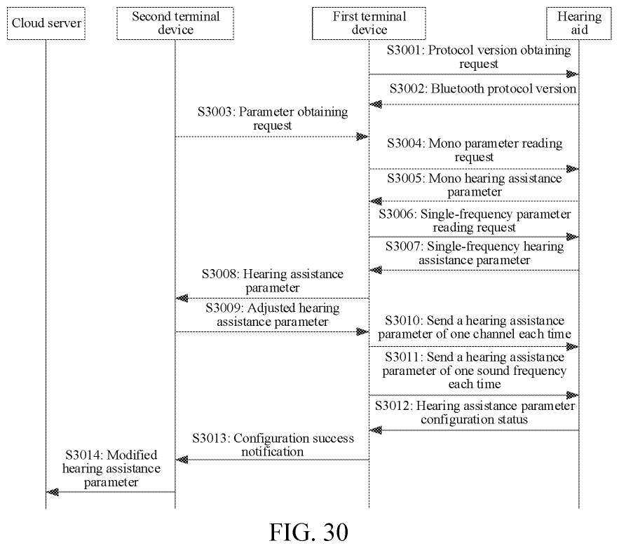

Referring to FIG. 30, the parameter modification includes operation S3001 to operation S3014, and details are described as follows:
Operation S3001: The first terminal device sends a protocol version obtaining request to the hearing assistance device.
Operation S3002: The hearing assistance device sends a Bluetooth protocol version to the first terminal device.

The hearing assistance device sends the Bluetooth protocol version of the hearing assistance device to the first terminal device according to the protocol version obtaining request.
Operation S3003: The second terminal device sends a parameter obtaining request to the first terminal device.

The parameter obtaining request is configured for obtaining a hearing assistance parameter of the hearing assistance device.
Operation S3004: The first terminal device sends a mono parameter reading request to the hearing assistance device.

If an MTU supported by the Bluetooth protocol version is relatively large, the first terminal device may read a hearing assistance parameter of one channel from the hearing assistance device each time, until all hearing assistance parameters of the hearing assistance device are obtained. Therefore, the first terminal device may send a mono parameter reading request configured for obtaining a hearing assistance parameter of one channel to the hearing assistance device.
Operation S3005: The hearing assistance device sends a mono hearing assistance parameter to the first terminal device.

The hearing assistance device sends a hearing assistance parameter of one channel to the first terminal device according to the mono parameter reading request.
Operation S3006: The first terminal device sends a single-frequency parameter reading request to the hearing assistance device.

If the MTU supported by the Bluetooth protocol version is relatively small, the first terminal device may read a hearing assistance parameter of one sound frequency from the hearing assistance device each time, until all hearing assistance parameters of the hearing assistance device are obtained. Therefore, the first terminal device may send the single-frequency parameter reading request configured for obtaining a hearing assistance parameter of one sound frequency to the hearing assistance device.
Operation S3007: The hearing assistance device sends a single-frequency hearing assistance parameter to the first terminal device.

The hearing assistance device sends a hearing assistance parameter of one sound frequency to the first terminal device according to the single-frequency parameter reading request.
Operation S3008: The first terminal device sends the hearing assistance parameter to the second terminal device.
Operation S3009: The second terminal device sends an adjusted hearing assistance parameter to the first terminal device.

The audiologist may adjust the hearing assistance parameter in the hearing assistance application of the second terminal device, to obtain the adjusted hearing assistance parameter, and send the adjusted hearing assistance parameter to the first terminal device.
Operation S3010: The first terminal device sends a hearing assistance parameter of one channel to the hearing assistance device each time.

If the MTU supported by the Bluetooth protocol version is relatively large, the first terminal device may send a hearing assistance parameter of one channel of modified hearing assistance parameters to the hearing assistance device each time, until all the modified hearing assistance parameters are sent to the hearing assistance device.
Operation S3011: The first terminal device sends a hearing assistance parameter of one sound frequency to the hearing assistance device each time.

If the MTU supported by the Bluetooth protocol version is relatively small, the first terminal device may send a hearing assistance parameter of one sound frequency of the modified hearing assistance parameters each time, until the modified hearing assistance parameters are all sent to the hearing assistance device.
Operation S3012: The hearing assistance device sends a hearing assistance parameter configuration status to the first terminal device.
Operation S3013: The second terminal device sends a configuration success notification to the first terminal device.

The configuration success notification is generated after the hearing assistance parameter configuration status indicates successful configuration.
Operation S3014: The second terminal device stores the modified hearing assistance parameter in the cloud server.

In the cloud server, hearing assistance parameters may be classified based on manufacturers of hearing assistance parameters, so that a manufacturer can conveniently obtain a hearing assistance parameter from the cloud server.

A header of a data packet transmitted between the hearing assistance device and the first terminal device includes a command and an instruction. The command includes a read command, a notification command, and a write command. Entering/exiting a hearing test mode, playing pure sound, obtaining a protocol version, reading a mono hearing assistance parameter, reading a single-frequency hearing assistance parameter, and the like correspond to the read command. Delivering a mono hearing assistance parameter, delivering a single-frequency hearing assistance parameter, and the like correspond to the write command. Periodical broadcast of electric quantity information, volume information, a hearing assistance device mode, and the like correspond to the notification command.
Process 1.7: View user information.

The audiologist may view the user information, for example, a user age, through a user information viewing service.

Specific implementation details of the operations in this embodiment are described in detail in the foregoing embodiments, and details are not described herein again.

In this embodiment, no high investment is needed in developing and maintaining an audio/video communication capability in the hearing assistance application, and the online conferencing application has stable communication. Therefore, communication quality during a hearing assistance service can be improved, web deployment of the hearing assistance application can be implemented, and a native application does not need to be updated in a process such as function upgrade, thereby improving ease of use. Data storage and a matching formula are performed in cloud, which can reduce dependency on performance of the terminal device. The audiologist can provide a hearing assistance service to the user without a high-performance device, and the audience group is wider. Based on point-to-point communication between the user and the audiologist, data transfer may be reduced, and real-time performance and data security may be improved. The connection between the hearing assistance device and the second terminal device is established by using a specified Bluetooth protocol, so that third-party access can be facilitated. As long as a manufacturer implements functions related to the protocol, a strong remote hearing test and matching capability can be implemented. For the audiologist, one application can implement the operation capability of hearing assistance devices of multiple brands.

In the foregoing solution, the hearing assistance terminal establishes the audio/video call with the assistance receiving terminal by using the call software. The call software herein has an audio/video call function, such as an online conferencing application or a social chat application. An establishment manner may be that the hearing assistance terminal initiates a request and the assistance receiving terminal answers. Alternatively, the assistance receiving terminal may initiate a request and the hearing assistance terminal answers. Then, the hearing assistance terminal obtains the execution instruction for the hearing assistance application hosted in the call software, where the instruction may be triggered by the hearing assistance provider, and then runs the hearing assistance application in the call software. Then, the data transmission channel with the assistance receiving terminal is establish based on the hearing assistance application, and is a duplex communication channel for transmitting application data of the hearing assistance application. The hearing assistance provider may trigger an invitation request at the hearing assistance terminal, and the assistance receiving terminal creates the channel after responding. The hearing assistance terminal starts to provide a hearing assistance service to the assistance receiving terminal through the data transmission channel and the audio/video call. In this solution, communication between two parties is implemented by using the audio/video call, thereby improving convenience and service accuracy, and the connection established based on the call software improves stability, and difficulty in developing the hearing assistance application is reduced, thereby improving application stability and service quality.

The hearing assistance terminal sends the test request to the assistance receiving terminal through the data transmission channel. The test request is configured to instruct the assistance receiving terminal to control the hearing assistance device to play a corresponding test audio. The test request may include related data of the test audio, for example, directly include the test audio, or include a test sound frequency and a test sound intensity. After receiving the request, the assistance receiving terminal controls the hearing assistance device to play the test audio, obtains feedback information representing whether the hearing assistance receiving party can hear the test audio, and sends the feedback information to the hearing assistance terminal. The hearing assistance terminal determines the hearing test result of the hearing assistance receiving party according to the feedback information, displays the result, and sends the result to the assistance receiving terminal for display. In this solution, the hearing test is performed on the hearing assistance receiving party through the data transmission channel and the audio/video call, thereby improving accuracy and convenience of the test.

When the hearing test result includes a hearing threshold of the hearing assistance receiving party at a specified test sound frequency, the hearing assistance terminal generates multiple test requests corresponding to the test sound frequency, where each request includes different test sound intensities and the same test sound frequency, and sequentially sends the requests to the assistance receiving terminal. During request generation, the feedback information of the current test request is obtained. If the condition for determining the hearing threshold is not satisfied, for example, the feedback information is insufficient, a test range is not covered, a sound intensity difference does not reach a threshold, playing of a test audio is problematic, or a state of the hearing assistance receiving party is unstable, a test sound intensity of a next request is calculated according to the current feedback information and the test sound intensity, and the next request is generated, until the hearing threshold is determined. This solution can automatically test hearing and adjust a test sound intensity according to the feedback, thereby improving hearing test efficiency.

In a process of generating the multiple test requests, the hearing assistance terminal obtains the feedback information of the current test request and obtains the maximum sound intensity and the minimum sound intensity configured for calculating the test sound intensity of the current test request. The maximum sound intensity and the minimum sound intensity are updated according to the feedback information. The maximum sound intensity is updated to a current test sound intensity if the feedback shows that the hearing assistance receiving party can hear the test audio; and the minimum sound intensity is updated if the hearing assistance receiving party cannot hear the test audio. If a difference between the updated maximum sound intensity and the updated minimum sound intensity is greater than or equal to the specified intensity threshold, a test sound intensity of a next test request is calculated according to the average value. If the difference is less than the specified intensity threshold, the hearing threshold at the test sound frequency is determined according to the average value. In this solution, the hearing threshold is gradually determined by using a bisection method, thereby further improving hearing test efficiency.

When sending multiple test requests, the hearing assistance terminal calculates the interval duration of the current test request. Interval durations of different requests are not completely the same, and may be determined by generating random numbers or by extracting according to a preset duration sequence. The transmission time of the next request is calculated according to the transmission time and the interval duration of the current request, and the next request is sent based on this time. This can avoid that a user finds a pattern due to periodical sending of test requests, thereby improving accuracy of the hearing test result.

After obtaining the hearing test result, the hearing assistance terminal stores the hearing test result in the server. The server may be a cloud server or a physical server. This operation reduces storage pressure of the hearing assistance terminal, and facilitates the hearing assistance provider in obtaining a result after logging into another terminal, thereby improving convenience.

When obtaining the hearing test result of the hearing assistance receiving party corresponding to the hearing assistance device connected to the assistance receiving terminal, the hearing assistance terminal may obtain the result through test or obtain a historical result from the server. The test result is sent to the server, and the server generates the parameter configuration policy for the hearing assistance device according to the result. The policy includes hearing assistance parameters at different sound frequencies. The hearing assistance terminal receives the policy, generates the parameter configuration request, and sends the parameter configuration request to the assistance receiving terminal through the data transmission channel. The assistance receiving terminal configures the hearing assistance parameter of the hearing assistance device according to the policy. In this solution, the policy is generated by the server, so that parameter configuration is accelerated, and calculation pressure of the hearing assistance terminal is reduced.

The hearing assistance terminal sends a parameter obtaining request to the assistance receiving terminal through the data transmission channel, the assistance receiving terminal obtains current hearing assistance parameters of the hearing assistance device and sends the current hearing assistance parameters to the hearing assistance terminal, and the hearing assistance terminal displays the parameters on the parameter adjustment interface. The hearing assistance provider triggers an adjustment operation on the interface. The hearing assistance terminal determines an adjusted target hearing assistance parameter, generates a parameter adjustment request, and sends the parameter adjustment request to the assistance receiving terminal. The assistance receiving terminal adjusts the hearing assistance parameter of the hearing assistance device according to the target parameter. In this solution, the parameter is adjusted through the data transmission channel and the audio/video call, thereby improving convenience and accuracy of parameter adjustment.

The assistance receiving terminal establishes an audio/video call with the hearing assistance terminal by using the call software, obtains the execution instruction for the hearing assistance application hosted in the call software, runs the application, and establishes the data transmission channel with the hearing assistance terminal based on the application. Through the channel and the audio/video call, the assistance receiving terminal obtains the hearing assistance service provided by the hearing assistance terminal. This solution also improves communication convenience, service accuracy and stability, and reduces application development difficulty.

The assistance receiving terminal establishes a connection to the hearing assistance device by using a target near field communication protocol such as a Bluetooth protocol. The control request sent by the hearing assistance terminal is received; multiple commands included in a specified protocol are searched for a corresponding target command; instruction groups corresponding to multiple hearing assistance services are searched for a target instruction group and a target instruction; a data packet is generated according to the control request, the target command, the target instruction group, and the target instruction, and the data packet is sent to the hearing assistance device. The hearing assistance device executes an instruction according to the data packet and the protocol. The hearing assistance service can be obtained as long as the hearing assistance device supports the specified protocol.

The assistance receiving terminal receives the protocol detection request sent by the hearing assistance terminal, obtains the near field communication protocol supported by the hearing assistance device, and sends the protocol to the hearing assistance terminal. The hearing assistance terminal compares the protocols, and sends an installation package of the specified protocol if the protocols do not match. The assistance receiving terminal sends the installation package to the hearing assistance device for installation, and establishes a connection to the hearing assistance device based on the set protocol after the installation. This solution can improve a success rate of the hearing assistance service, and ensure that the hearing assistance device supports the required protocol.

The assistance receiving terminal receives the parameter configuration request sent by the hearing assistance terminal, where the request includes multiple hearing assistance parameters. The target manner is selected from multiple parameter configuration manners, and the hearing assistance parameters are grouped according to the manner, to obtain multiple parameter groups. Different configuration manners correspond to different grouping and may be selected according to a version of the near field communication protocol. A data packet corresponding to the parameter group is generated and sent to the hearing assistance device. The hearing assistance device performs parameter configuration according to a parameter in the data packet. This solution improves flexibility of parameter configuration.

When the multiple hearing assistance parameters include hearing assistance parameters respectively corresponding to the left channel and the right channel, and each channel includes hearing assistance parameters at multiple sound frequencies, the assistance receiving terminal may group hearing assistance parameters of the same channel into one group according to the target parameter configuration manner, to obtain parameter groups of the left channel and the right channel, or may group hearing assistance parameters of the same channel and the same sound frequency into one group, to obtain multiple parameter groups. Therefore, flexibility of parameter grouping and configuration is further improved.

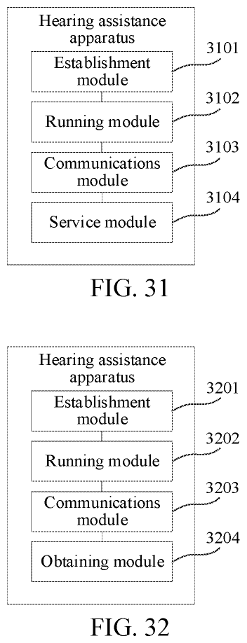

FIG. 31 is a block diagram of a hearing assistance apparatus according to an exemplary embodiment of the present disclosure. As shown in FIG. 31, the apparatus includes:
an establishment module 3101, configured to establish an audio/video call between a hearing assistance terminal and an assistance receiving terminal by using call software;
a running module 3102, configured to obtain an execution instruction for a hearing assistance application integrated into the call software, and run the hearing assistance application in the call software according to the execution instruction;
a communications module 3103, configured to establish a data transmission channel between the hearing assistance terminal and the assistance receiving terminal based on the hearing assistance application, the data transmission channel being a duplex communication channel for transmitting application data of the hearing assistance application; and
a service module 3104, configured to provide a hearing assistance service to the assistance receiving terminal through the data transmission channel and the audio/video call.

In an exemplary embodiment, based on the foregoing solution, the service module 3104 is specifically configured to:
transmit a test request to the assistance receiving terminal through the data transmission channel, and determine, according to feedback information corresponding to the test request, a hearing test result of a hearing assistance receiving party corresponding to a hearing assistance device, the test request being configured to instruct the assistance receiving terminal to control the hearing assistance device to play a test audio corresponding to the test request, and the feedback information representing whether the hearing assistance receiving party can hear the test audio; and
display the hearing test result and transmitting the hearing test result to the assistance receiving terminal, so that the assistance receiving terminal displays the hearing test result.

In an exemplary embodiment, based on the foregoing solution, if the hearing test result includes a hearing threshold of the hearing assistance receiving party at a specified test sound frequency, the service module 3104 is specifically configured to:
generate multiple test requests corresponding to the test sound frequency, and transmit the multiple test requests to the assistance receiving terminal through the data transmission channel; each test request including a test sound intensity and the test sound frequency, and different test requests including different test sound intensities; and
in a process of generating the multiple test requests, obtain feedback information corresponding to a current test request; calculate, in response to determining, according to the feedback information corresponding to the current test request, that a condition for determining the hearing threshold of the hearing assistance receiving party at the test sound frequency is not satisfied, a corresponding test sound intensity of a next test request according to the feedback information and a corresponding test sound intensity of the current test request; and generate the next test request according to the corresponding test sound intensity of the next test request and the test sound frequency, until the hearing threshold of the hearing assistance receiving party at the test sound frequency is determined.

In an exemplary embodiment, based on the foregoing solution, the service module 3104 is specifically configured to:
obtain a maximum sound intensity and a minimum sound intensity for the current test request; the corresponding test sound intensity of the current test request being less than or equal to the maximum sound intensity and being greater than or equal to the minimum sound intensity;
update the maximum sound intensity and the minimum sound intensity according to the feedback information corresponding to the current test request; where if the feedback information corresponding to the current test request indicates that the hearing assistance receiving party can hear the test audio corresponding to the current test request, the maximum sound intensity is updated to the corresponding test sound intensity of the current test request; and if the feedback information corresponding to the current test request indicates that the hearing assistance receiving party cannot hear the test audio corresponding to the current test request, the minimum sound intensity is updated to the corresponding test sound intensity of the current test request; and
if a difference between the updated maximum sound intensity and the updated minimum sound intensity is greater than or equal to a specified intensity threshold, calculate the corresponding test sound intensity of the next test request according to an average value of the updated maximum sound intensity and the updated minimum sound intensity; or
if the difference between the updated maximum sound intensity and the updated minimum sound intensity is less than the specified intensity threshold, determine the hearing threshold at the test sound frequency according to the average value of the updated maximum sound intensity and the updated minimum sound intensity.

In an exemplary embodiment, based on the foregoing solution, the service module 3104 is specifically configured to:
calculate an interval duration of the current test request; where interval durations corresponding to different test requests are not completely the same;
calculate a transmission time of the next test request according to a transmission time and the interval duration of the current test request; and
transmit the next test request to the assistance receiving terminal through the data transmission channel according to the transmission time of the next test request.

In an exemplary embodiment, based on the foregoing solution, the apparatus further includes a storage module, configured to:
store the hearing test result into a server; where the server includes a cloud server.

In an exemplary embodiment, based on the foregoing solution, the service module 3104 is specifically configured to:
obtain a hearing test result of a hearing assistance receiving party corresponding to a hearing assistance device connected to the assistance receiving terminal;
transmit the hearing test result to a server, so that the server generates, according to the hearing test result, a parameter configuration policy for the hearing assistance device;
receive the parameter configuration policy transmitted by the server, and generate a parameter configuration request according to the parameter configuration policy; and
transmit the parameter configuration request to the assistance receiving terminal through the data transmission channel, so that the assistance receiving terminal configures a hearing assistance parameter of the hearing assistance device according to the parameter configuration policy.

In an exemplary embodiment, based on the foregoing solution, the service module 3104 is specifically configured to: send a parameter obtaining request to the assistance receiving terminal through the data transmission channel, so that the assistance receiving terminal obtains a current hearing assistance parameter of the hearing assistance device according to the parameter obtaining request;
receive, through the data transmission channel, the current hearing assistance parameter transmitted by the assistance receiving terminal, and display the current hearing assistance parameter on a parameter adjustment interface;
determine, in response to an adjustment operation triggered on the parameter adjustment interface, a target hearing assistance parameter obtained after the current hearing assistance parameter is adjusted, and generate a parameter adjustment request according to the target hearing assistance parameter; and
transmit the parameter adjustment request to the assistance receiving terminal through the data transmission channel, so that the assistance receiving terminal adjusts a hearing assistance parameter of the hearing assistance device according to the target hearing assistance parameter.

The hearing assistance apparatus provided in FIG. 31 and the hearing assistance method corresponding to the hearing assistance terminal provided in the foregoing embodiment belong to the same idea. Specific manners in which the modules and units perform operations have been described in detail in the method embodiments. Details are not described herein again.

FIG. 32 is a block diagram of a hearing assistance apparatus according to an exemplary embodiment of the present disclosure. As shown in FIG. 32, the apparatus includes:
an establishment module 3201, configured to establish an audio/video call between an assistance receiving terminal and a hearing assistance terminal by using call software;
a running module 3202, configured to obtain an execution instruction for a hearing assistance application integrated into the call software, and run the hearing assistance application in the call software according to the execution instruction;
a communications module 3203, configured to establish a data transmission channel between the assistance receiving terminal and the hearing assistance terminal based on the hearing assistance application, the data transmission channel being a duplex communication channel for transmitting application data of the hearing assistance application; and
an obtaining module 3204, configured to obtain, through the data transmission channel and the audio/video call, a hearing assistance service provided by the hearing assistance terminal.

In an exemplary embodiment, based on the foregoing solutions, the obtaining module 3204 is specifically configured to:
establish a connection between the assistance receiving terminal and a hearing assistance device based on a target near field communication protocol;
receive, through the data transmission channel, a control request transmitted by the hearing assistance terminal, and search multiple commands included in the target near field communication protocol for a target command corresponding to the control request; where the multiple commands include at least one of a read command, a write command, or a notification command;
search instruction groups respectively corresponding to multiple hearing assistance services included in the target near field communication protocol for a target instruction group corresponding to a hearing assistance service to which the control request belongs, and search the target instruction group for a target instruction corresponding to the control request; where the multiple hearing assistance services include at least one of a hearing test service, a matching service configured to update a hearing assistance parameter of the hearing assistance device, and an information management service configured to manage attribute information of the hearing assistance device;
generate, according to the control request, the target command, the target instruction group, and the target instruction, a data packet corresponding to the control request; where a header of the data packet sequentially includes identification information of the target command, identification information of the target instruction group, and identification information of the target instruction; and
transmit the data packet to the hearing assistance device through the connection, so that the hearing assistance device executes the target instruction according to the data packet and the target near field communication protocol.

In an exemplary embodiment, based on the foregoing solutions, the obtaining module 3204 is specifically configured to:
receive, through the data transmission channel, a protocol detection request transmitted by the hearing assistance terminal, and obtain, according to the protocol detection request, a near field communication protocol supported by the hearing assistance device;
transmit the near field communication protocol supported by the hearing assistance device to the hearing assistance terminal through the data transmission channel, so that the hearing assistance terminal compares the near field communication protocol supported by the hearing assistance device with the target near field communication protocol;
receive, through the data transmission channel, an installation package that is transmitted by the hearing assistance terminal and that corresponds to the target near field communication protocol, and transmit the installation package to the hearing assistance device, so that the hearing assistance device is installed with the installation package; where the installation package is transmitted after the hearing assistance terminal detects that the near field communication protocol supported by the hearing assistance device does not match the target near field communication protocol; and
establish a connection to the hearing assistance device based on the target near field communication protocol.

In an exemplary embodiment, based on the foregoing solutions, the obtaining module 3204 is specifically configured to:
receive, through the data transmission channel, a parameter configuration request transmitted by the hearing assistance terminal; where the parameter configuration request includes multiple hearing assistance parameters of the hearing assistance device;
select a target parameter configuration manner corresponding to the parameter configuration request from multiple parameter configuration manners, and group the multiple hearing assistance parameters according to the target parameter configuration manner, to obtain multiple parameter groups; where different parameter configuration manners correspond to different grouping manners;
generate data packets respectively corresponding to the multiple parameter groups, to obtain multiple data packets; where a data packet corresponding to each parameter group includes a hearing assistance parameter in the parameter group; and
transmit the multiple data packets to the hearing assistance device, so that the hearing assistance device configures a hearing assistance parameter of the hearing assistance device according to the hearing assistance parameter included in the received data packet.

In an exemplary embodiment, the multiple hearing assistance parameters include hearing assistance parameters respectively corresponding to a left channel and a right channel, and a hearing assistance parameter corresponding to each channel includes hearing assistance parameters respectively corresponding to multiple sound frequencies, based on the foregoing solution, the obtaining module 3204 is specifically configured to:
group hearing assistance parameters that belong to the same channel and that are of the multiple hearing assistance parameters into the same group according to the target parameter configuration manner, to obtain parameter groups respectively corresponding to the left channel and the right channel;
or
group hearing assistance parameters that belong to the same channel and the same sound frequency and that are of the multiple hearing assistance parameters into the same group according to the target parameter configuration manner, to obtain the multiple parameter groups.

The hearing assistance apparatus provided in FIG. 32 and the hearing assistance method corresponding to the assistance receiving terminal provided in the foregoing embodiment belong to the same idea. Specific manners in which the modules and units perform operations have been described in detail in the method embodiments. Details are not described herein again.

An embodiment of the present disclosure further provides an electronic device, including one or more processors; and a storage apparatus, configured to store one or more computer programs, the one or more computer programs, when executed by the one or more processors, causing the electronic device to implement the hearing assistance method according to the foregoing embodiments.

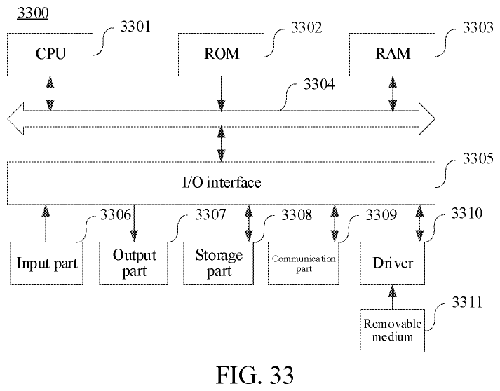

FIG. 33 is a schematic structural diagram of a computer system adapted to implement an electronic device according to an embodiment of the present disclosure.

A computer system 3300 of the electronic device shown in FIG. 33 is merely an example, and does not constitute any limitation on functions and use ranges of the embodiments of the present disclosure.

As shown in FIG. 33, the computer system 3300 includes a central processing unit (CPU) 3301, which may perform various suitable actions and processing based on a computer program stored in a read-only memory (ROM) 3302 or a computer program loaded from a storage part 3308 into a random access memory (RAM) 3303, for example, perform the hearing assistance method described in the foregoing embodiments. The RAM 3303 further stores various computer programs and data required for system operations. The CPU 3301, the ROM 3302, and the RAM 3303 are connected to each other through a bus 3304. An input/output (I/O) interface 3305 is also connected to the bus 3304.

In some embodiments, the following components are connected to the I/O interface 3305: an input part 3306 including a keyboard, a mouse, or the like, an output part 3307 including a cathode ray tube (CRT), a liquid crystal display (LCD), a speaker, or the like, a storage part 3308 including a hard disk, or the like, and a communication part 3309 including a network interface card such as a local area network (LAN) card or a modem. The communication part 3309 performs communication processing by using a network such as the Internet. A driver 3310 is also connected to the I/O interface 3305 as required. A removable medium 3311 such as a magnetic disk, an optical disk, a magneto-optical disk, or a semiconductor memory is installed on the drive 3310 as required, so that a computer program read from the removable medium is installed into the storage part 3308 as required.

Particularly, according to an embodiment of the present disclosure, a computer program for implementing the hearing assistance method may be carried in a computer readable medium, and the computer program may be downloaded and installed from a network by using the communication part 3309 and/or installed from the removable medium 3311.

The computer-readable medium shown in the embodiments of the present disclosure may be a computer-readable signal medium or a computer-readable storage medium or any combination of the two. The computer-readable storage medium may be, for example, an electric, magnetic, optical, electromagnetic, infrared, or semi-conductive system, apparatus, or device, or any combination of the above. A more specific example of the computer-readable storage medium may include but is not limited to: an electrical connection having one or more wires, a portable computer magnetic disk, a hard disk, a random access memory (RAM), a read-only memory (ROM), an erasable programmable read-only memory (EPROM), a flash memory, an optical fiber, a compact disk read-only memory (CD-ROM), an optical storage device, a magnetic storage device, or any appropriate combination thereof. In the present disclosure, the computer-readable storage medium may be any tangible medium containing or storing a computer program, and the computer program may be used by or used in combination with an instruction execution system, an apparatus, or a device. The computer readable signal medium may include a data signal propagated in a baseband or as a part of a carrier, and carries a computer readable computer program. The propagated data signal may use a multiple forms, which includes, but is not limited to, an electromagnetic signal, an optical signal, or any suitable combination thereof. The computer program included in the computer-readable medium may be transmitted by using any suitable medium, including but not limited to: a wireless medium, a wire, or the like, or any suitable combination thereof.

The flowcharts and block diagrams in the accompanying drawings illustrate example system architectures, functions and operations that may be implemented by a system, a method, and a computer program product according to various embodiments of the present disclosure. Each box in a flowchart or a block diagram may represent a module, a program segment, or a part of code. The module, the program segment, or the part of code includes one or more executable instructions configured for implementing specified logic functions. The term module (and other similar terms such as submodule, unit, subunit, etc.) in this disclosure may refer to a software module, a hardware module, or a combination thereof. A software module (e.g., computer program) may be developed using a computer programming language. A hardware module may be implemented using processing circuitry and/or memory. Each module can be implemented using one or more processors (or processors and memory). Likewise, a processor (or processors and memory) can be used to implement one or more modules. Moreover, each module can be part of an overall module that includes the functionalities of the module. In some implementations used as substitutes, functions annotated in boxes may alternatively occur in a sequence different from that annotated in an accompanying drawing. For example, actually two boxes shown in succession may be performed basically in parallel, and sometimes the two boxes may be performed in a reverse sequence. This is determined by a related function. Each box in a block diagram or a flowchart and a combination of boxes in the block diagram or the flowchart may be implemented by using a dedicated hardware-based system configured to perform a specified function or operation, or may be implemented by using a combination of dedicated hardware and a computer program.

A related unit described in the embodiments of the present disclosure may be implemented in a software manner, or may be implemented in a hardware manner, and the unit described can also be set in a processor. Names of the units do not constitute a limitation on the units in a specific case.

According to another aspect of the present disclosure, a computer-readable storage medium is further provided, having a computer program stored therein. The computer program, when executed by a processor of an electronic device, causes the electronic device to implement the hearing assistance method as described above. The computer-readable storage medium may be included in the electronic device described in the foregoing embodiments, or may exist alone without being installed into the electronic device.

According to another aspect of the present disclosure, a computer program product is further provided, including a computer program, the computer program, when executed by a processor, implementing the hearing assistance method as described above. The computer program may be stored in a computer readable storage medium. The computer program product may be a computer program used as a product, for example, an application (APP), a web page, or a mini program. Alternatively, the computer program product may be a storage medium, a device, a terminal, a virtual machine, or the like including a computer program.

Technical features of the foregoing embodiments may be combined in various manners. To make description concise, not all possible combinations of the technical features in the foregoing embodiments are described. However, the combinations of these technical features shall be considered as falling within the scope recorded by this specification provided that no conflict exists.

The foregoing embodiments only describe several implementations of the present disclosure, which are described specifically and in detail, but cannot be construed as a limitation to the patent scope of the present disclosure. For a person of ordinary skill in the art, several transformations and improvements can be made without departing from the idea of the present disclosure. These transformations and improvements belong to the protection scope of the present disclosure. Therefore, the protection scope of the patent of the present disclosure shall be subject to the appended claims.

What is claimed is:

1. A hearing assistance method, executed by a hearing assistance terminal, comprising:
establishing an audio/video call between the hearing assistance terminal and an assistance receiving terminal by using call software;
obtaining an execution instruction for a hearing assistance application integrated into the call software, and running the hearing assistance application in the call software according to the execution instruction;
establishing a data transmission channel between the hearing assistance terminal and the assistance receiving terminal based on the hearing assistance application, the data transmission channel being a duplex communication channel for transmitting application data of the hearing assistance application; and
providing a hearing assistance service to the assistance receiving terminal through the data transmission channel and the audio/video call.

2. The method according to claim 1, wherein the providing a hearing assistance service to the assistance receiving terminal through the data transmission channel and the audio/video call comprises:
transmitting a test request to the assistance receiving terminal through the data transmission channel, and determining, according to feedback information corresponding to the test request, a hearing test result of a hearing assistance receiving party corresponding to a hearing assistance device, the test request being configured to instruct the assistance receiving terminal to control the hearing assistance device to play a test audio corresponding to the test request, and the feedback information representing whether the hearing assistance receiving party can hear the test audio; and
displaying the hearing test result and transmitting the hearing test result to the assistance receiving terminal.

3. The method according to claim 2, wherein the hearing test result comprises a hearing threshold of the hearing assistance receiving party at a test sound frequency; and
the transmitting a test request to the assistance receiving terminal through the data transmission channel, and determining, according to feedback information corresponding to the test request, a hearing test result of a hearing assistance receiving party corresponding to a hearing assistance device comprises:
generating multiple test requests corresponding to the test sound frequency, and transmitting the multiple test requests to the assistance receiving terminal through the data transmission channel; a test request comprising a test sound intensity and the test sound frequency, and different test requests comprising different test sound intensities; and
in a process of generating the multiple test requests, obtaining feedback information corresponding to a current test request; calculating, in response to determining, according to the feedback information corresponding to the current test request, that a condition for determining the hearing threshold of the hearing assistance receiving party at the test sound frequency is not satisfied, a corresponding test sound intensity of a next test request according to the feedback information and a corresponding test sound intensity of the current test request; and generating the next test request according to the corresponding test sound intensity of the next test request and the test sound frequency, until the hearing threshold of the hearing assistance receiving party at the test sound frequency is determined.

4. The method according to claim 3, wherein the calculating, in response to determining, according to the feedback information corresponding to the current test request, that a condition for determining the hearing threshold of the hearing assistance receiving party at the test sound frequency is not satisfied, a corresponding test sound intensity of a next test request according to the feedback information and a corresponding test sound intensity of the current test request comprises:
obtaining a maximum sound intensity and a minimum sound intensity for the current test request; the corresponding test sound intensity of the current test request being less than or equal to the maximum sound intensity and being greater than or equal to the minimum sound intensity;
updating the maximum sound intensity and the minimum sound intensity according to the feedback information corresponding to the current test request; wherein in response to the feedback information corresponding to the current test request indicating that the hearing assistance receiving party can hear the test audio corresponding to the current test request, the maximum sound intensity is updated to the corresponding test sound intensity of the current test request; and in response to the feedback information corresponding to the current test request indicating that the hearing assistance receiving party cannot hear the test audio corresponding to the current test request, the minimum sound intensity is updated to the corresponding test sound intensity of the current test request; and
in response to a difference between the updated maximum sound intensity and the updated minimum sound intensity being greater than or equal to an intensity threshold, calculating the corresponding test sound intensity of the next test request according to an average value of the updated maximum sound intensity and the updated minimum sound intensity; or
in response to the difference between the updated maximum sound intensity and the updated minimum sound intensity being less than the intensity threshold, determining the hearing threshold at the test sound frequency according to the average value of the updated maximum sound intensity and the updated minimum sound intensity.

5. The method according to claim 3, wherein the sequentially transmitting the multiple test requests to the assistance receiving terminal through the data transmission channel comprises:
calculating an interval duration of the current test request; wherein interval durations corresponding to at least two test requests are not the same;
calculating a transmission time of the next test request according to a transmission time and the interval duration of the current test request; and
transmitting the next test request to the assistance receiving terminal through the data transmission channel according to the transmission time of the next test request.

6. The method according to claim 2, further comprising:
storing the hearing test result into a server; wherein the server comprises a cloud server.

7. The method according to claim 1, wherein the providing a hearing assistance service to the assistance receiving terminal through the data transmission channel and the audio/video call further comprises:
obtaining a hearing test result of a hearing assistance receiving party corresponding to a hearing assistance device connected to the assistance receiving terminal;
transmitting the hearing test result to a server;
receiving a parameter configuration policy generated and transmitted by the server based on the hearing test result, and generating a parameter configuration request according to the parameter configuration policy; and
transmitting the parameter configuration request to the assistance receiving terminal through the data transmission channel, a hearing assistance parameter of the hearing assistance device being configured according to the parameter configuration policy.

8. The method according to claim 1, wherein the providing a hearing assistance service to the assistance receiving terminal through the data transmission channel and the audio/video call comprises:
transmitting a parameter obtaining request to the assistance receiving terminal through the data transmission channel;
receiving, through the data transmission channel, a current hearing assistance parameter of the hearing assistance device transmitted by the assistance receiving terminal in response to the parameter obtaining request, and displaying the current hearing assistance parameter on a parameter adjustment interface;
determining, in response to an adjustment operation triggered on the parameter adjustment interface, a target hearing assistance parameter obtained after the current hearing assistance parameter is adjusted, and generating a parameter adjustment request according to the target hearing assistance parameter; and
transmitting the parameter adjustment request to the assistance receiving terminal through the data transmission channel, a hearing assistance parameter of the hearing assistance device being adjusted according to the target hearing assistance parameter.

9. An electronic device, comprising:
one or more processors; and
a storage apparatus, configured to store one or more computer programs, the one or more computer programs, when executed by the one or more processors, causing the electronic device to implement the method according to claim 1.

10. A non-transitory computer-readable storage medium, having a computer program stored therein, the computer program, when executed by a processor, causing the processor to implement the method according to claim 1.

11. A hearing assistance method, performed by an assistance receiving terminal, comprising:
establishing an audio/video call between the assistance receiving terminal and a hearing assistance terminal by using call software;
obtaining an execution instruction for a hearing assistance application integrated into the call software, and running the hearing assistance application in the call software according to the execution instruction;
establishing a data transmission channel between the assistance receiving terminal and the hearing assistance terminal based on the hearing assistance application, the data transmission channel being a duplex communication channel for transmitting application data of the hearing assistance application; and
obtaining, through the data transmission channel and the audio/video call, a hearing assistance service provided by the hearing assistance terminal.

12. The method according to claim 11, wherein the obtaining, through the data transmission channel and the audio/video call, a hearing assistance service provided by the hearing assistance terminal comprises:
establishing a connection between the assistance receiving terminal and a hearing assistance device based on a near field communication protocol;
receiving, through the data transmission channel, a control request transmitted by the hearing assistance terminal, and searching multiple commands comprised in the near field communication protocol for a target command corresponding to the control request; wherein the multiple commands comprise at least one of a read command, a write command, or a notification command;
searching instruction groups respectively corresponding to multiple hearing assistance services comprised in the near field communication protocol for a target instruction group corresponding to a hearing assistance service to which the control request belongs, and searching the target instruction group for a target instruction corresponding to the control request; wherein the multiple hearing assistance services comprise at least one of a hearing test service, a matching service configured to update a hearing assistance parameter of the hearing assistance device, and an information management service configured to manage attribute information of the hearing assistance device;
generating, according to the control request, the target command, the target instruction group, and the target instruction, a data packet corresponding to the control request; wherein a header of the data packet sequentially comprises identification information of the target command, identification information of the target instruction group, and identification information of the target instruction; and
transmitting the data packet to the hearing assistance device through the connection, so that the hearing assistance device executes the target instruction according to the data packet and the target near field communication protocol.

13. The method according to claim 12, wherein the establishing a connection between the assistance receiving terminal and a hearing assistance device based on the target near field communication protocol comprises:
receiving, through the data transmission channel, a protocol detection request transmitted by the hearing assistance terminal, and obtaining, according to the protocol detection request, a near field communication protocol supported by the hearing assistance device;
transmitting the near field communication protocol supported by the hearing assistance device to the hearing assistance terminal through the data transmission channel, to be compared with the near field communication protocol supported by the hearing assistance device;
in response to detecting the near field communication protocol supported by the hearing assistance device does not match the target near field communication protocol, receiving, through the data transmission channel, an installation package that is transmitted by the hearing assistance terminal and that corresponds to the target near field communication protocol, and transmitting the installation package to the hearing assistance device for installation; and
establishing a connection to the hearing assistance device based on the target near field communication protocol.

14. The method according to claim 11, wherein the obtaining, through the data transmission channel and the audio/video call, a hearing assistance service provided by the hearing assistance terminal comprises:
receiving, through the data transmission channel, a parameter configuration request transmitted by the hearing assistance terminal; wherein the parameter configuration request comprises multiple hearing assistance parameters of the hearing assistance device;
selecting a target parameter configuration manner corresponding to the parameter configuration request from multiple parameter configuration manners, and grouping the multiple hearing assistance parameters according to the target parameter configuration manner, to obtain multiple parameter groups; wherein different parameter configuration manners correspond to different grouping manners;
generating data packets respectively corresponding to the multiple parameter groups, to obtain multiple data packets; wherein a data packet corresponding to a parameter group comprises a hearing assistance parameter in the parameter group; and
transmitting the multiple data packets to the hearing assistance device for configuration of the hearing assistance parameter.

15. The method according to claim 14, wherein the multiple hearing assistance parameters comprise hearing assistance parameters respectively corresponding to a left channel and a right channel, and a hearing assistance parameter corresponding to each channel comprises hearing assistance parameters respectively corresponding to multiple sound frequencies; and
the grouping the multiple hearing assistance parameters according to the target parameter configuration manner, to obtain multiple parameter groups comprises:
grouping hearing assistance parameters that belong to the same channel and that are of the multiple hearing assistance parameters into the same group according to the target parameter configuration manner, to obtain parameter groups respectively corresponding to the left channel and the right channel.

16. The method according to claim 14, wherein the multiple hearing assistance parameters comprise hearing assistance parameters respectively corresponding to a left channel and a right channel, and a hearing assistance parameter corresponding to each channel comprises hearing assistance parameters respectively corresponding to multiple sound frequencies; and
the grouping the multiple hearing assistance parameters according to the target parameter configuration manner, to obtain multiple parameter groups comprises:
grouping hearing assistance parameters that belong to the same channel and the same sound frequency and that are of the multiple hearing assistance parameters into the same group according to the target parameter configuration manner, to obtain the multiple parameter groups.

17. An electronic device, comprising:
one or more processors; and
a storage apparatus, configured to store one or more computer programs, the one or more computer programs, when executed by the one or more processors, causing the electronic device to implement the method according to claim 11.

18. A non-transitory computer-readable storage medium, having a computer program stored therein, the computer program, when executed by a processor, causing the processor to implement:
establishing an audio/video call between the assistance receiving terminal and a hearing assistance terminal by using call software;
obtaining an execution instruction for a hearing assistance application integrated into the call software, and running the hearing assistance application in the call software according to the execution instruction;
establishing a data transmission channel between the assistance receiving terminal and the hearing assistance terminal based on the hearing assistance application, the data transmission channel being a duplex communication channel for transmitting application data of the hearing assistance application; and
obtaining, through the data transmission channel and the audio/video call, a hearing assistance service provided by the hearing assistance terminal.

19. The storage medium according to claim 18, wherein the obtaining, through the data transmission channel and the audio/video call, a hearing assistance service provided by the hearing assistance terminal comprises:
establishing a connection between the assistance receiving terminal and a hearing assistance device based on a near field communication protocol;
receiving, through the data transmission channel, a control request transmitted by the hearing assistance terminal, and searching multiple commands comprised in the near field communication protocol for a target command corresponding to the control request; wherein the multiple commands comprise at least one of a read command, a write command, or a notification command;
searching instruction groups respectively corresponding to multiple hearing assistance services comprised in the near field communication protocol for a target instruction group corresponding to a hearing assistance service to which the control request belongs, and searching the target instruction group for a target instruction corresponding to the control request; wherein the multiple hearing assistance services comprise at least one of a hearing test service, a matching service configured to update a hearing assistance parameter of the hearing assistance device, and an information management service configured to manage attribute information of the hearing assistance device;
generating, according to the control request, the target command, the target instruction group, and the target instruction, a data packet corresponding to the control request; wherein a header of the data packet sequentially comprises identification information of the target command, identification information of the target instruction group, and identification information of the target instruction; and
transmitting the data packet to the hearing assistance device through the connection, so that the hearing assistance device executes the target instruction according to the data packet and the target near field communication protocol.

20. The storage medium according to claim 19, wherein the establishing a connection between the assistance receiving terminal and a hearing assistance device based on the target near field communication protocol comprises:
receiving, through the data transmission channel, a protocol detection request transmitted by the hearing assistance terminal, and obtaining, according to the protocol detection request, a near field communication protocol supported by the hearing assistance device;
transmitting the near field communication protocol supported by the hearing assistance device to the hearing assistance terminal through the data transmission channel, to be compared with the near field communication protocol supported by the hearing assistance device;
in response to detecting the near field communication protocol supported by the hearing assistance device does not match the target near field communication protocol, receiving, through the data transmission channel, an installation package that is transmitted by the hearing assistance terminal and that corresponds to the target near field communication protocol, and transmitting the installation package to the hearing assistance device for installation; and
establishing a connection to the hearing assistance device based on the target near field communication protocol.
